(19) United States
(12) Patent Application Publication
(10) Pub. No.: US 2005/0196862 A1
(43) Pub. Date: Sep. 8, 2005

(54) DNA cassette for cellular expression of small RNA

(76) Inventors: Christine I. Wooddell, Madison, WI (US); Hans Herweijer, Madison, WI (US); Jon A. Wolff, Madison, WI (US)

Correspondence Address: MIRUS CORPORATION, 505 SOUTH ROSA RD, MADISON, WI, 53719, US

(21) Appl. No.: 10/652,971

(22) Filed: Aug. 29, 2003

Related U.S. Application Data
(60) Provisional application No. 60/407,103, filed on Aug. 30, 2002.
(60) Provisional application No. 60/436,198, filed on Dec. 23, 2002.
(60) Provisional application No. 60/475,588, filed on Jun. 4, 2003.

(51) Int. Cl. A61K 48/00; C12N 15/85

(52) U.S. Cl. 435/455; 514/44

(57) ABSTRACT

We describe expression cassettes and processes for preparing expression cassettes that can be delivered to animal cells in vivo or in vitro. Delivery of the cassettes results in expression of small RNA transcripts such as siRNA in the cell. The cassettes can by used to inhibit gene expression in the cell and to screen siRNA and other RNA sequences for functional efficacy.

### CROSS-REFERENCE TO RELATED APPLICATIONS

This application claims priority benefit of U.S. Provisional Application Ser. No. 60/407,103 filed Aug. 30, 2002 and U.S. Provisional Application Ser. No. 60/436,198 filed Dec. 23, 2002 and U.S. Provisional Application Ser. No. 60/475,588 filed Jun. 4, 2003.

### BACKGROUND OF THE INVENTION

Recently, there has been a great deal of research interest in the delivery of RNA oligonucleotides to cells, due to the discovery of RNA interference. It has been shown that through the delivery of small double stranded RNA's, greater than 80% knockdown of endogenous gene expression levels can be obtained, without inhibiting the expression of non-targeted genes. RNA interference (RNAi) describes the phenomenon whereby the presence of double-stranded RNA (dsRNA) of sequence that is identical or highly similar to a target gene results in the degradation of messenger RNA (mRNA) transcribed from that target gene [Sharp 2001]. It has been found that RNAi in mammalian cells is mediated by short interfering RNAs (siRNAs) of approximately 21-25 nucleotides in length [Tuschl et al. 1999 and Elbashir et al. 2001].

The ability to specifically inhibit expression of a target gene by RNAi has obvious benefits. For example, RNAi could be used to study gene function. In addition, RNAi could be used to inhibit the expression of deleterious genes and therefore alleviate symptoms of or cure disease or infection. For example, genes contributing to a cancerous state or to viral replication could be inhibited. In addition, mutant genes causing dominant genetic diseases such as myotonic dystrophy could be inhibited. Inflammatory diseases such as arthritis could also be treated by inhibiting such genes as cyclooxygenase or cytokines. Examples of targeted organs would include the liver, pancreas, spleen, skin, brain, prostrate, heart etc. In addition, RNAi could be used to generate animals that mimic true genetic “knockout” animals to study gene function.

Drug discovery and target validation in pharmaceutical research could also be facilitated by siRNA technology. Information for drug targeting will be gained not only by inhibiting a potential drug target but also by determining whether an inhibited protein, and therefore the pathway, has significant phenotypic effects. For example, inhibition of LDL receptor expression should raise plasma LDL levels and, therefore, suggest that up-regulation of the receptor would be of therapeutic benefit. Expression arrays can be used to determine the responsive effect of inhibition on the expression of genes other than the targeted gene or pathway [Sharp 2001]. It will place the gene product within functional pathways and networks (interacting pathways).

The observation of gene silencing in mammalian cells after delivering synthetic dsRNA molecules into them has revolutionized the study of gene function. The idea is simple: design 21-mer siRNAs against the target mRNA of interest and study the phenotype of the cells after delivering the siRNA. Due to its simplicity, the approach provides a tool for large-scale, high throughput analysis of mammalian genes. This makes siRNA an excellent instrument in target gene validation for drug discovery.

Gene silencing has a great therapeutic potential. In many inherited and acquired diseases the cause of the disease is the production of a protein from a mutant endogenous gene, from a viral gene, or the overproduction of a protein normally present at low quantities. Also, therapeutic intervention in diseases may also be mediated by lowering the expression of specific genes in biochemical pathways. The efficacy of antisense RNA in inhibiting protein production from pathogenic mRNAs has been explored for decades, and has provided some promising therapeutic applications.

In order to provide long-term excess, the antisense RNA can be produced within the cell as a transcript from an expression cassette (or multiple expression cassettes) transfected into the cell. Gene silencing by RNAi is very similar to the antisense approach in the sense of post-transcriptional regulation and target specificity, but has the advantage of requiring less RNA molecules per cell due to the involvement of an enzymatic, catalytic mechanism. A single siRNA molecule can be used over and over again to initiate the cleavage and destruction of multiple mRNA targets. Thus, any disease, in which the inhibition (or knock-down) of a well defined protein(s) may have a therapeutic effect, is a good candidate for siRNA gene therapy [Tuschl and Borkhardt 2002].

Synthetic siRNA duplexes (typically between 19-30 base pairs in length) can be designed and generated against any gene of known sequence. There are some guidelines and software that make designing siRNAs easier. In spite of the guidelines, not all sequences are equally efficient in initiating degradation of a target mRNA. The best, most effective siRNAs have to be determined empirically. The synthetic siRNA then has to be delivered into the cytoplasm by one of various delivery methods, such as transfection using liposomes or polymers.

An alternative approach to the delivery of synthetically produced oligonucleotides is the construction of expression cassettes that will generate siRNA within the cell. The currently used siRNA expression cassettes take advantage of RNA Polymerase III (Pol-III) promoters, such as human U6 (hU6), human H1 (hH1), mouse U6 (mU6), tRNA^{Val} and VA2. Other siRNA expression vectors with RNA Polymerase II (Pol-II) promoters such as the U1 small nuclear RNA and the cytomegalovirus (CMV) promoters have also been described. These promoters drive expression of short RNA transcripts that can become siRNA. Transcripts produced by RNA Polymerase-III lack the polyA tail and have well defined transcription start and termination signals. The termination signal for Pol-III is a run of 4-6 thymidines, some of which are transcribed. The expression cassette can be designed to yield a short RNA resembling the synthetic siRNA with overhanging 3′ nucleotides. An siRNA molecule is formed from short complementary sense and anti-sense strands of RNA. The sense strand of the siRNA has the same sequence as a short region of the mRNA targeted for RNAi. The siRNA strands may originate from different transcripts expressed in the same cell or both siRNA strands may be part of a single transcript that folds back to form a short hairpin structure [Paddison and Hannon 2002]. The expression cassette(s) encoding the siRNA strands can be delivered to cells in the same fragment of DNA (hairpin expression cassette) or on separate fragments of DNA (sense and anti-sense strand expression cassettes). Thus, the two basic types of siRNA expression constructs code either for a hairpin RNA containing both the sense and the antisense sequence, separated by a loop region, or they contain two separate promoters driving the transcription of the sense and antisense RNA strands independently.

### SUMMARY OF THE INVENTION

In a preferred embodiment we describe a process for inhibiting gene expression in a cell though RNA interference by the delivery of expression cassettes comprising small fragments of DNA wherein the fragments comprise: a promoter and a sequence encoding an RNA function inhibitor. Optionally, the expression cassettes may also contain a transcription termination sequence and a 3′ extension that improves the efficacy of the cassette. The expression cassettes can be generated by PCR amplification from a promoter template and specially designed PCR primers. The forward PCR primer is upstream of or within the promoter. The RNA function inhibitor sequence, such as an siRNA, is encoded in one or more downstream primers. Multiple expression cassettes can be generated easily and quickly to screen multiple RNA function inhibitor sequences for activity. The overall length of the linear expression cassette can vary from a couple hundred base-pairs to over 1,000 bp. A preferred promoter is an RNA polymerase III promoter.

In a preferred embodiment we describe expression cassettes containing a promoter, a sequence for a desired functional RNA, a transcription termination sequence and a 3′ extension. No additional sequences are necessary. Depending on the promoter, the cassette may be less than 300 base pairs in length. The addition of a 3′ extension downstream of the transcription termination signal improves the efficacy of the expression cassettes. The 3′ extension is any nucleotide sequence that is present in the cassette and is downstream of the transcription termination signal. A preferred promoter is an RNA polymerase III promoter. The expression cassettes can be delivered to a cell in vitro or in vivo. Delivery of the cassette to the cell results in production of the functional RNA in the cell. The functional RNA can be selected from the group consisting of: siRNA, an anti-sense RNA, a microRNA, a ribozyme and an aptamer. The production of the functional RNA in the cell can result in sequence-specific inhibition of a gene. The inhibited gene can be a transfected gene, a recombinant gene, a transgene, an endogenous gene or a viral or bacterial gene.

In a preferred embodiment, we describe a process for improving expression from a linear expression cassette comprising: incorporating a 3′ extension onto the cassette downstream of a transcription termination signal. Compared to expression cassettes which contain no bases beyond the transcription termination signal, expression cassettes containing a 3′ extension exhibit improved function. The 3′ extension is any nucleotide sequence that is present in the cassette and is downstream of the transcription termination signal. A preferred 3′ extension contains 15 or more nucleotides.

In a preferred embodiment we describe expression cassettes for expressing functional RNAs in a cell comprising: PCR products amplified from promoter templates using an upstream PCR primer and a downstream primer wherein the downstream primer contains the template sequence for the RNA of interest. The upstream primer anneals to the template upstream of or within the promoter sequence. The downstream primer is designed to add a coding sequence for the functional RNA to the PCR product downstream of the promoter. Optionally the downstream primer may also add a transcription termination signal and a 3′ extension onto the PCR product. The expression cassettes may be as small as 130 bp and may be up to 1000 bp or longer. The PCR generated expression cassette can be delivered to a cell wherein the RNA is expressed. For RNA function inhibitors, such as siRNA, expression of the RNA can result in inhibition of gene expression.

In a preferred embodiment, we describe a process for inhibiting gene expression in an animal cell comprising: delivering to the cell a linear double stranded DNA 100 to 500 base pairs in length from which an siRNA is transcribed. The siRNA may be a hairpin siRNA transcribed from a single DNA or the siRNA may be composed of a sense strand and an anti-sense strand transcribed from separate DNAs and which anneal in the cell. The DNA may be generated by any process known in the art. Most notably, the DNA can be generated by PCR amplification from a template. The template may contain the entire sequence that is to be delivered to the cell or a portion of the sequence incorporated into the linear DNA may be derived from a PCR primer.

In a preferred embodiment, the described expression cassettes and processes can be used to screen potential RNA sequences to identify those the provide optimal activity. Many expression cassettes, to express many different functional RNAs (such as siRNA), can be made more quickly and at lower cost compared to synthetically producing the RNA, according to the invention. The described expression cassettes can also be generated more quickly than traditional cloning into plasmids.

In a preferred embodiment, the cell can be an animal cell that is maintained in tissue culture such as cell lines that are immortalized or transformed. These include a number of cell lines that can be obtained from American Type Culture Collection (Bethesda) such as, but not limited to: 3T3 (mouse fibroblast) cells, Rat1 (rat fibroblast) cells, CHO (Chinese hamster ovary) cells, CV-1 (monkey kidney) cells, COS (monkey kidney) cells, 293 (human embryonic kidney) cells, HeLa (human cervical carcinoma) cells, HepG2 (human hepatocytes) cells, Sf9 (insect ovarian epithelial) cells and the like.

In another preferred embodiment, the cell can be a primary or secondary cell which means that the cell has been maintained in culture for a relatively short time after being obtained from an animal. These include, but are not limited to, primary liver cells and primary muscle cells and the like. The cells within the tissue are separated by mincing and digestion with enzymes such as trypsin or collagenases which destroy the extracellular matrix. Tissues consist of several different cell types and purification methods such as gradient centrifugation or antibody sorting can be used to obtain purified amounts of the preferred cell type. For example, primary myoblasts are separated from contaminating fibroblasts using Percoll (Sigma) gradient centrifugation.

In another preferred embodiment, the cell can be an animal cell that is within the tissue in situ or in vivo meaning that the cell has not been removed from the tissue or the animal.

In a preferred embodiment, a kit is provided comprising: reagents required for the preparation and transfection of siRNA expression cassettes. The kit includes vectors containing promoter templates, upstream promoter PCR primers, control primers and a transfection reagent. The kit can be used to generate expression cassettes for desired RNAs, such as siRNA, and for testing siRNA sequences for efficacy. Instructions for use are also provided with the kit. By the term instructions for use, it is meant a tangible expression describing the reagent concentration for at least one assay method, parameters such as the relative amount of reagent and sample to be admixed, maintenance time periods for reagent/sample admixtures, temperature, buffer conditions and the like.

Further objects, features, and advantages of the invention will be apparent from the following detailed description when taken in conjunction with the accompanying drawings.

### BRIEF DESCRIPTION OF THE FIGURES

FIG. 1. Schematic for formation and use of siRNA expression cassettes. The schematic shows two methods for generating siRNA: a) separate expression cassettes for transcribing the sense and anti-sense siRNA strands; or, b) generating a short hairpin expression cassette by two sequential reactions (on the left).

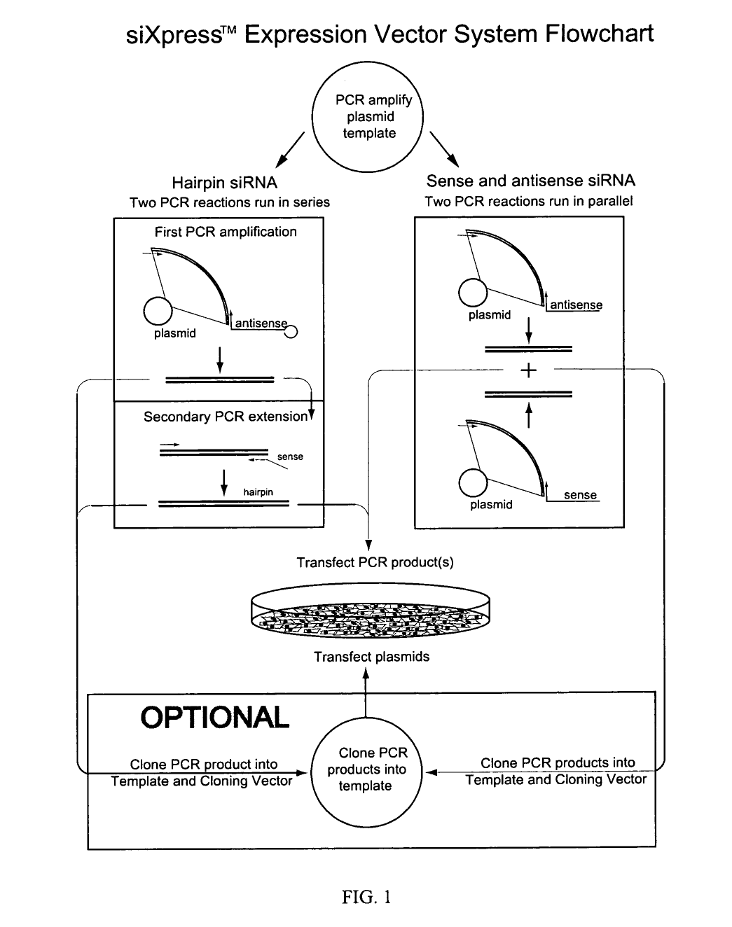

FIG. 2. Schematic of expression cassettes to separately generate sense and an anti-sense RNA oligonucleotides.

FIG. 3. An example of an expression cassette design to separately generate the sense and anti-sense strands of an siRNA targeting the firefly luc+mRNA sequence SEQ ID 2.

FIG. 4. Schematic of expression cassettes for generating short hairpin RNAs. Two designs are shown. The upper design codes for a hairpin that is anti-sense RNA, loop, sense RNA. The lower design codes for a sense, loop, anti-sense hairpin.

FIG. 5. Example of a hairpin expression cassette designed to generate an siRNA targeting the firefly luc+mRNA.

FIG. 6. Illustrations of the siXpress™ Vectors.

FIG. 7. Bar graph illustrating induction of RNA interference mediated by small expression cassettes. CHO-Luc cells were transfected with 0.2, 0.5 or 1.0 μg of a 525 bp PCR fragment containing a cassette for expression of a luciferase hairpin siRNA or with synthetic siRNA oligonucleotides. Expression is scaled to that in cells treated with transfection reagent only and is shown as percent expression.

FIG. 8. Bar graph illustrating induction of RNA interference in a cell line stably expressing a transgene. Small PCR generated expression cassettes are as effective as plasmids for expression of functional siRNAs. Luciferase expression is shown for each sample scaled to the empty vector sample as 100%.

FIG. 9. Bar graph illustrating induction of RNA interference in HeLa cells cotransfected with PCR generated expression cassettes and reporter gene plasmids. The firefly luc+/Renilla luc ratio from the cell lysate of each sample was normalized to this ratio from cells transfected with the empty vector. Values are presented as percentage of normalized expression.

FIG. 10. Bar graph illustrating that small amounts of PCR generated hairpin siRNA expression cassette mediate RNAi. Luciferase expression is shown for each sample scaled to the PCR fragment expressing the SEAP siRNA, which was set as 100%.

FIG. 11. Bar graph illustrating improved function of hairpin expression cassettes with 3′ extensions of 25 or more bases. The firefly luc+/Renilla luc ratio from the cell lysate of each sample was normalized to this ratio from cells transfected with the U6 vector pMIR219. Values are presented as percentage of normalized expression.

FIG. 12. Bar graph illustrating improved induction of RNA interference when 3′ extensions are added to the siRNA expression cassettes. U6 hairpin cassettes were made with downstream extensions of 0-25 bp. CHO-Luc cells were transfected with hU6-sh1-luc expression cassettes with 0, 5, 10, 15, 20, or 25 bp extensions added downstream of the termination signal (bars labeled 0, 5, 10, 15, 20, and 25); pMIR225, EcoRI/ori3 383 bp PCR product (hU6-luc plasmid PCR); pMIR232, mU6-HindIII/DL46 PCR product (mU6-luc plasmid PCR); pMIR225 (hU6-luc plasmid); and pMIR232 (mU6-luc plasmid). Expression of each sample is shown in relative light units. Error bars indicate the standard deviation from triplicate wells.

FIG. 13. Bar graph illustrating improved efficacy of separate strand expression cassettes when downstream extensions are used. The sizes of the PCR expression cassettes (in bp) are shown above the bars in the graph. The luc cassettes had +0, +20, +40 or +62 bp 3′ extensions as indicated under the bars. Cell lysates were assayed for firefly and Renilla luciferase. The mean ratio from all wells transfected with SEAP siRNA cassettes was set as 100%. All samples were normalized to this firefly luc/Renilla luciferase ratio.

FIG. 14. Bar graph illustrating screening siRNA sequences for activity using separate strand siRNA expression cassettes. Expression of SEAP in each sample was scaled to expression in the wells transfected with the control separate strand luc cassettes with +20 extensions.

FIG. 15. Bar graph illustrating knockdown of endogenous lamin mRNA using lamin hairpin siRNA expression cassettes. Lamin mRNA was quantitated by Q-PCR relative to input RNA concentration (dark grey bars) or relative to GAPDH concentration (light grey bars). Expression is scaled to the average of cells that were transfected with the control siRNA cassette.

FIG. 16. Bar graph illustrating effective knockdown of gene expression in vivo using PCR generated hairpin siRNA expression cassettes. Expression in each sample (n=4) was measured as the firefly luc+/Renilla ratio and normalized to that ratio of reporter plasmids only samples.

FIG. 17. Bar graph illustrating PCR-based siRNA expression cassette knockdown of target gene expression in vivo. Expression in each sample (n=4) was measured as the firefly luc+/Renilla ratio and normalized to that ratio of reporter plasmids only samples.

FIG. 18. Bar graph illustrating function of small human H1 promoter expression cassettes in vivo. Expression in each sample (n=4) was measured as the firefly luc+/Renilla ratio and normalized to that ratio of reporter plasmids only samples.

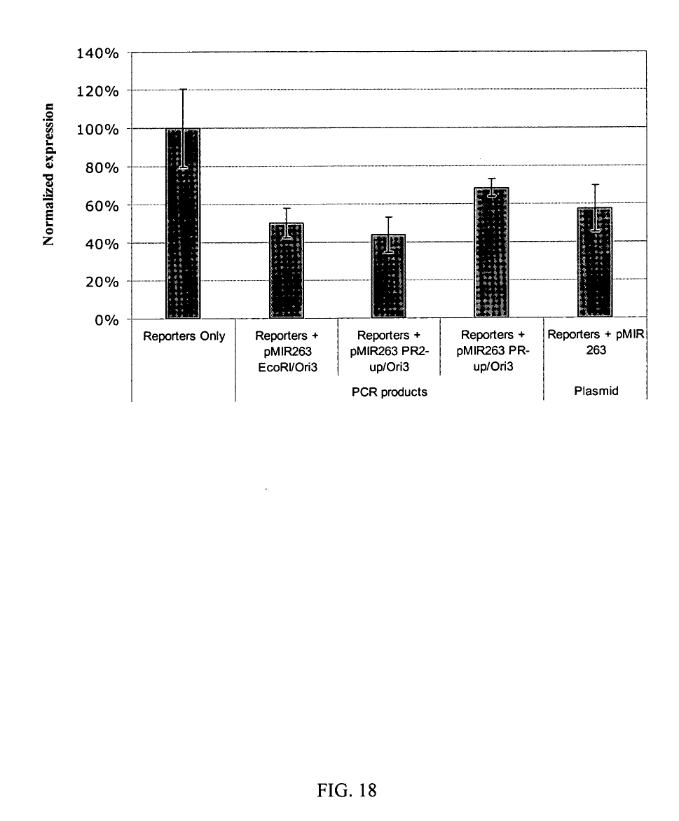

FIG. 19. Bar graph illustrating long-term target gene knock-down by siXpress cassettes in vivo. SEAP reporter plasmid and SEAP-specific or control expression vectors were delivered to mice via tail vein injection. Knockdown on SEAP expression was observed with SEAP siRNA expression vectors 42 days post injection.

FIG. 20. Bar graph illustrating in vivo long-term RNA interference mediated by siRNA expression vector. SiRNA-specific inhibition of gene expression was observed out to 42 days in mice transfected with SEAP separate strand siRNA expression vectors but not luc+separate strand siRNA expression vectors. SEAP expression of each group was averaged and is shown at the indicated times post injection. Expression is shown as ng SEAP/ml serum.

### DETAILED DESCRIPTION OF THE INVENTION

We describe a method for expressing an RNA in a cell by delivering to the cell an expression cassette comprising small fragments of DNA. The expression cassettes can be generated by PCR amplification of a promoter template using specially designed primers. The forward, or upstream, PCR primer anneals to the template upstream of the transcription initiation site and upstream of or within the promoter region. The promoter consists of any sequence to which an RNA polymerase binds and initiates transcription of the DNA. The promoter may be an RNA Polymerase I promoter, an RNA polymerase II promoter or an RNA polymerase III promoter. An example of an acceptable promoter is the RNA Polymerase III U6 promoter. The upstream primer may have additional sequence at the 5′ end such as a restriction enzyme recognition site. The reverse, or downstream, PCR primer is composed of several regions comprising: a template annealing sequence, a sequence of interest, a transcription termination sequence and a 3′ extension sequence. The template annealing sequence consists of sequence that anneals to the template and serves as a primer for DNA synthesis by a DNA polymerase.

The template annealing sequence typically encompasses position −1 relative to the transcription start site and extends upstream of the start site. Position −1 is the nucleotide immediately upstream of the first nucleotide transcribed by the RNA polymerase. The first nucleotide transcribed by the RNA polymerase, position +1, may also be encompassed by the annealing sequence if this nucleotide is present in the template. The sequence of interest consists of the DNA sequence that is to be transcribed into an RNA in the cell. The sequence may be selected from the group consisting of: sense strand of an siRNA, anti-sense strand, siRNA hairpin sequence, microRNA, antisense RNA, ribozyme, aptamer, or other RNA. The transcription termination sequence consists of any signal that causes transcription termination. For an RNA Polymerase III promoter, an example of a terminator is a run of 4 or more adenosines in the template strand (4 or more thymidines in the coding or non-template strand). The addition of a 3′ extension downstream of the transcription termination signal improves the efficacy of the expression cassettes. The downstream primer may have additional sequence at the 5′ end of the primer such as a restriction enzyme site. Using the two described PCR primers to amplify the template, a small expression cassette is generated which contains a promoter that drives expression of a desired RNA sequence. The overall length of the linear DNA fragment containing an siRNA expression cassette can vary from a about a hundred base-pairs to over 1,000 bp. DNA fragments that encode an siRNA expression cassette can be transfected directly into cells in vitro or in vivo.

It is also possible to generate the expression cassette using sequential PCR reactions. This method is especially useful when more than 50 nucleotides are to be added downstream of the transcription start site in the expression cassette. For sequential PCR reactions downstream primers which contain overlapping sections of the template annealing sequence, sequence of interest, transcription termination sequence and 3′ extension sequence may be used in sequential reactions. For example, in a first round of PCR amplification, the downstream primer (the first downstream primer) may consist of the template annealing sequence and the sequence of interest. In a second round or PCR amplification, using the product from the first round as a template, the sequence at the 3′ end of the second downstream primer would be identical to the sequence at the 5′ end of the first downstream primer over a sufficient length to serve as a primer for DNA synthesis by a DNA polymerase. The second downstream primer sequence would then also consist of the transcription termination sequence and 3′ extension sequence, thereby extending the length of the expression cassette. Any number of sequential PCR amplification steps may be used to generate the expression cassette. The same upstream primer may be used for all the PCR amplification steps.

We have found that 3′ extensions improve the efficacy of small linear RNA expression cassettes. In referring to a small RNA expression cassette, a 3′ extension is any nucleotide sequence that is present downstream of the promoter and of the RNA coding sequence in the expression cassette. If the expression cassette includes a transcription termination signal, then the extension is the nucleotide sequence downstream of the termination signal. The termination signal itself is not considered part of the 3′ extension. For RNA polymerase III expression cassettes, the termination signal is a run of 4 or more thymidines in the coding (non-template) strand of the expression cassette, coded for by the 4 or more adenosines in the template strand. A 3′ extension is any added nucleotides that extends the length of the cassette at the 3′ end and improves the efficacy of the RNA expression cassette. The length of the 3′ extension required for improved efficacy of the siRNA expression cassette can be dependent on the promoter sequence, the coding sequence and the target cell. A simple and appropriate method for testing extension lengths is to include an extension sequence on the 5′ end of the downstream PCR primer used for generating the expression cassette by PCR. The extensions can be added in one PCR reaction or using multiple PCR reactions. For example, a PCR product that has a shorter 3′ extension may be used as the template in subsequent PCR reactions to add longer 3′ extensions. The first PCR reaction to generate a sense strand siRNA or anti-sense strand siRNA expression cassette, for example, can include a 20 base 3′ extension sequence at the 5′ end of the downstream primer. The PCR product from this reaction can be further amplified in a second reaction with another downstream primer which anneals to the 20 base extension sequence from the first reaction. This second primer would have the first 20 base extension sequence at the primer's 3′ end and would have additional bases at the 5′ end of the primer. Alternatively, if the expression cassette is present in another polynucleotide such as a plasmid, different downstream PCR primers which anneal at different distances downstream of the coding sequence of the cassette may be used, or the linear expression cassette can be derived by release from the plasmid DNA sequence by restriction enzyme digestion.

Typically an extension of 25 bp would be a sufficient length to improve expression from an expression cassette. However, for specific cassettes, smaller 3′ extensions may be sufficient or longer 3′ extensions may be optimal. The specific sequence of the 3′ extension is not critically important to its function. Several sequences have been used successfully. A number of sequences are expected to function as long as the extension is of the appropriate length.

The cassette is typically generated by PCR amplification or enzyme digestion of a longer polynucleotide and is double-stranded. However, for other methods of generating the expression cassette, a single-stranded extension of either the transcribed or the non-transcribed strand may also be functional for improving expression cassette function.

These small RNA expression cassettes enable the production of functional RNAs in a cell without the need for cloning the desired coding sequence into a plasmid and without the cost of synthesizing RNA. We show that the expression cassettes function in cells in vitro and in vivo. Using the described invention, multiple expression cassettes can be generated easily and quickly to screen RNA sequences for activity.

Fragments of DNA differ from plasmids in that there are terminal ends. The 5′ ends of the fragment can be covalently altered by enzymatic or chemical reaction or can have non-covalent attachments. A fragment of DNA generated by PCR can have attachments at the ends by having a modified terminus on the PCR primer or by having modifications anywhere in a primer that was used for the PCR reaction. Modified bases could be inserted in the PCR fragment during the PCR reaction. Modified bases can also be inserted in effective siRNA cassette-containing fragments of DNA using other kinds of DNA polymerases, such as T7 DNA polymerase or Klenow enzyme. The primers that are used for PCR can have many kinds of modifications. The modifications can stabilize the polynucleotide, allow the polynucleotide to be followed in the cell or body of a mammal, or impart some other useful property to the polynucleotide, such as targeting. Some of these include modified bases, chemically reactive groups, fluorescent or radioactive molecules, cross-linking moieties, and biotinylated nucleotides. Ligands can be attached to the ends. One method to do so is through the biotinylated nucleotide. Ligands allow for targeting of the siRNA expression cassettes to specific tissues or cancer cells, for example. In summary, a fragment of DNA containing an siRNA expression cassette can have many kinds of alterations that increase its utility. Many of the options that can be applied to fragments of DNA would not be available for use with plasmids or synthetic siRNA.

The expression cassettes can be delivered to a cell by any transfection agent or delivery system known in the art. The expression cassettes can be delivered to a cell in vitro or in vivo to deliver siRNA for inhibition of gene expression. The cassettes may be used to produce small hairpin RNA that functions as siRNA to induce RNA interference. This type of cassette is referred to as a hairpin cassette. Alternatively, the expression cassettes may by used in pairs wherein the sense strand of an siRNA in transcribed from one cassette and the complementary anti-sense strand of an siRNA is transcribed from a second expression cassette; i.e. separate strand siRNA expression cassettes. The expression cassettes can also be delivered to a cell in vitro or in vivo to deliver other functional RNAs.

RNA polymerase II expression cassettes for siRNA can be generated from a vector that includes an enhancer/promoter transcription control region template and a downstream primer that encodes the sequence of interest. For RNA polymerase II promoter templates, run-off transcription can be used [Gou 2003]. The RNA polymerase II cassette does not require downstream extensions, but it can function with a short polyadenylation sequence [Miller 2003]. We have developed expression vectors that have short enhancer/promoter regions (500-700 bp) and give high levels of sustained expression in the liver. These vectors include the albumin promoter and enhancer elements originating from the intergenic region between the albumin and alpha-fetoprotein genes. Fragments of DNA with these enhancer/promoters can be used to mediate liver-specific RNAi.

Some of the effective siRNA expression cassettes discovered using this invention were cloned into a plasmid vector with a filler sequence that has allowed the siRNA to be expressed in vivo for at least 6 weeks with greater than 100-fold knock-down of target gene expression. The filler sequence is of mammalian origin from the albumin gene locus and has a very low CpG content. Other fragments of mammalian origin from a transcriptionally active region of the genome (euchromatin) with suppressed CpG content (less than the 1/16 statistical frequency) would be expected to have the same effect as the filler sequence used. The filler fragment could be used with any Pol-II or Pol-III promoter.

The PCR-based procedure for siRNA expression allows for an easy and fast method for testing siRNA cassette designs. The cassettes can be tested in cells in culture or in vivo. The DNA fragments can be transfected, electroporated, or micro-injected into cells in culture. The cassettes can be delivered to animals by any procedure for delivering DNA. Delivery methods include hydrodynamic procedures, electroporation, and delivery by lipid reagents or particles. One such method for delivery to mouse liver is increased pressure tail vein injection.

The target mRNA can be measured using Northern blotting, primer extension, quantitative RT-PCR, the Invader Assay (Third Wave, Madison, Wis.), or microarrays, among other methods. The protein expressed from the target gene can be detected by Western blotting, immunohistochemistry, or functional assays, among other methods.

The cassettes can be co-delivered with a reporter gene that allows transfected cells to be evaluated apart from untransfected cells. Green fluorescent protein (GFP) is an example of such a reporter gene. It allows the cells that received the DNA (including the expression cassettes) to be isolated by flow cytometry or to be evaluated in situ. Another example of a reporter gene that allows transfected cells to be isolated is a receptor gene that can tag the cells and allow them to be isolated by interaction with an antibody. One such example is the truncated human CD4 receptor utilized by Miltenyi Biotec (Auburn, Calif.). The methods for isolating or separately evaluating cells that received reporter genes and expression cassettes can be applied to cells in vivo. Cells in tissues that receive the expression cassette can be marked by co-delivery with any reporter gene that can be detected in situ.

An expression cassette can be tested by delivery into cells into which the target gene is also delivered. For example, the cDNA for a target human gene can be cloned into a plasmid vector or delivered in another form for expression in a cell in which it is not normally expressed. This target gene could be co-delivered with a number of expression cassettes of different designs to test them for efficacy. Alternatively, the target RNA could be prepared in a test tube or from a separate source and co-delivered with expression cassettes.

The siRNA expression cassettes can be utilized to knock down target gene expression in cultured cells to study gene function. The siRNA expression cassettes can be used for drug discovery and target validation. The expression cassettes survive longer in cells than synthetic siRNA.

Smaller fragments of DNA may be delivered more efficiently into some cells than plasmids. The PCR-based procedure for generating siRNA expression cassettes can be used to generate material for delivery to cells. Alternatively, the method can be used to determine the parameters of an effective expression cassette, such as an optimal siRNA design and suitable 3′ extension lengths. The cassette can be cloned into a plasmid vector or other vector.

We further describe siXpress™ PCR Vector Systems which contain reagents required for the optimized preparation and transfection of siRNA expression cassettes. Three siXpress™ PCR Vector Systems, each utilizing a different polymerase III promoter: human U6, mouse U6, and human H1 are described. Double-stranded DNA containing the siRNA expression cassette is generated by polymerase chain reaction (PCR) and transfected into mammalian cells in vitro using Trans IT®-LT1 Transfection Reagent. Following nuclear transcription of the siRNA expression cassette, siRNA transcripts are exported into the cytoplasm and enter the RNAi pathway. RNAi applications include verification of effective siRNA sequences, studies of gene function, drug development, target validation, and other biological studies. The optimal PCR-generated expression cassettes can also be cloned into the supplied Template/Cloning Vector and grown under kanamycin selection in bacteria to provide a large amount of plasmid for further gene silencing experiments. The system can also be used for production of other small RNAs.

Each siXpress™ PCR Vector System provides sufficient materials to generate 20 siRNA expression cassettes. The supplied Luciferase Control Vector generates an siRNA hairpin directed against the firefly luciferase (luc+) gene. PCR products generated from this vector can be used as negative controls in experiments with user-designed siRNA expression cassettes. The Control Vector can also be used as a positive control in cells transiently or stably expressing luciferase.

Benefits of the siXpress system include: a) Enables efficient in situ expression of siRNA and knockdown; b) Synthesize multiple siRNA expression cassettes by PCR in hours; c) Select the siRNA expression cassette that provides the most desirable effect; d) Option of cloning PCR generated expression cassette in supplied vector; and, e) Complete Systems: provided with primers, template, controls and Trans IT®-LT1 Transfection Reagent.

Components of each siXpress Mouse U6, Human U6 or Human H1 promoter PCR Vector System kit include: upstream promoter primer, a kit matching promoter template vector, downstream sequencing primer, a kit matching promoter Luciferase control template vector, and TransIT-LT1 Transfection Reagent.

### DEFINITIONS

Expression cassette: The term expression cassette refers to a natural or recombinantly produced nucleic acid molecule that is capable of expressing a gene or genetic sequence. An expression cassette typically includes a promoter (allowing transcription initiation), and a sequence encoding one or more proteins. Optionally, the expression cassette may include transcriptional enhancers, non-coding sequences, splicing signals, transcription termination signals, and polyadenylation signals. Optionally, the expression cassette may include translation termination signals, a polyadenosine sequence, internal ribosome entry sites (IRES), and non-coding sequences. Optionally, the expression cassette may include a gene or partial gene sequence that is not translated into a protein. A nucleic acid can be used to modify the genomic or extrachromosomal DNA sequences. This can be achieved by delivering a nucleic acid that is expressed. Alternatively, the nucleic acid can effect a change in the DNA or RNA sequence of the target cell. This can be achieved by hybridization, multi-strand nucleic acid formation, homologous recombination, gene conversion, RNA interference or other yet to be described mechanisms.

The promoter of the cassette is defined to be upstream of the sequences that serve as the transcriptional template for generating small inhibitory RNA strands. The template strand of the expression cassette is defined as the strand of DNA that is used by RNA polymerase as a template for RNA synthesis. The non-template strand is the strand of the DNA that is anti-parallel to the template strand. The non-template strand of the DNA is also called the coding strand because it contains identical sequence to the transcribed RNA.

The term gene generally refers to a nucleic acid sequence that comprises coding sequences necessary for the production of a nucleic acid (e.g., siRNA) or a polypeptide or precursor. The term encompasses the coding region of a gene and includes sequences located adjacent to the coding region on both the 5′ and 3′ ends for a distance of up to 1 kb or more on either end. The term gene encompasses synthetic, recombinant, cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature RNA transcript. The term gene also refers to other regions or sequences including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotides) or at distant sites (more than 10,000 nucleotides). These non-coding sequences influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenic. One example of covalent modification of nucleic acid involves the action of LabelIT reagents (Mirus Corporation, Madison, Wis.).

As used herein, the term “gene expression” refers to the process of converting genetic information encoded in a gene into RNA (e.g., small RNA, siRNA, mRNA, rRNA, tRNA, or snRNA) through transcription of a deoxyribonucleic gene (e.g., via the enzymatic action of an RNA polymerase). Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

A small expression cassette is an expression cassette that is designed to produce, in a cell, an RNA transcript of 15 to 1000 bases. The cassette may produce an RNA that forms a transcript containing a secondary structure, such as a hairpin, ribozyme, or aptamer. Alternatively, the cassette may produce a transcript that base pairs with another transcript such as an endogenous mRNA or a transcript from another small RNA expression cassette. An example of a small RNA expression cassette is a cassette that produces an siRNA. The siRNA may be a hairpin siRNA or it may be formed by the base pairing of an expressed sense strand RNA and an expressed antisense strand RNA. The expression cassette may also produce an anti-sense RNA.

Functional RNA: A functional RNA comprises any RNA that is not translated into protein but whose presence in the cell alters the endogenous properties of the cell.

RNA function inhibitor: A RNA function inhibitor comprises any polynucleotide or nucleic acid analog containing a sequence whose presence or expression in a cell causes the degradation of or inhibits the function or translation of a specific cellular RNA, usually an mRNA, in a sequence-specific manner. Inhibition of RNA can effectively inhibit expression of a gene from which the RNA is transcribed. RNA function inhibitors are selected from the group comprising: siRNA, microRNA, interfering RNA, dsRNA, RNA Polymerase II transcribed DNAs encoding siRNA or antisense genes, RNA Polymerase III transcribed DNAs encoding siRNA or antisense genes, ribozymes, and antisense nucleic acid. SiRNA comprises a double stranded structure typically containing 15-50 base pairs and preferably 21-29 base pairs and having a nucleotide sequence identical or nearly identical to an expressed target gene or RNA within the cell. An siRNA may be composed of two annealed polynucleotides or a single polynucleotide that forms a hairpin structure. Antisense RNAs comprise sequence that is complimentary to an mRNA. Antisense polynucleotides include, but are not limited to: RNA, morpholinos, 2′-O-methyl polynucleotides, DNA, and the like. RNA polymerase III transcribed DNAs contain promoters selected from the list comprising: U6 promoters, H1 promoters, and tRNA promoters. RNA polymerase II promoters include U1, U2, U4, and U5 promoters, snRNA promoters, microRNA promoters, and mRNA promoters. These DNAs can be delivered to a cell wherein the DNA is transcribed to produce small hairpin siRNAs, separate sense and anti-sense strand linear siRNAs, antisense RNA or ribozymes.

A hairpin siRNA is an RNA transcript that includes a segment of the sense strand of the target mRNA and also its complement, the anti-sense strand. Following transcription, the RNA folds back on itself to form a hairpin structure. The sense and anti-sense strands are separated by 4 to 23 bases that form the loop of the hairpin. The hairpin RNA can be in either orientation, anti-sense-loop-sense or sense-loop-anti-sense. Loops can be any sequence. They may include a restriction enzyme site. MicroRNA loop sequences are favored because they may allow more of the nuclear-transcribed hairpin to be exported to the [Kawasaki and Taira 2003]. Examples of loop sequences are 5′-CTTCCTGTCA-3′ (SEQ ID 9) from the human mir-23 transcript and 5′-TTCAAGAGA-3′ (SEQ ID 10) from the Caenorhabditis elegans let-7 transcript. The loop of the hairpin transcript is cut off by the enzyme Dicer in mammalian cells, thereby forming the double-stranded siRNA. Dicer may trim the non-loop end of the hairpin as well. The siRNA strands of the hairpin are each typically 19 to 29 bases long [Paddison and Hannon 2002], thus forming a 19 to 29 base-paired stem. The requirements for the first base of the transcript are determined by the polymerase that will transcribe the expression cassette. RNA polymerase III transcribes mouse and human U6 promoters and requires the first transcribed base to be guanine (G). Transcription from the human H1 promoter can start with any of the bases, but adenine (A) is the starting nucleotide for the endogenous promoter and may be preferred. For expression cassettes from other promoters, the first base of the transcript would preferably be the normal +1 base for that particular promoter. The RNA polymerase III termination signal is a run of 5-6 thymidine residues immediately following the siRNA sequence. Some or all of the termination signal is likely to be transcribed, resulting in 2-6 uridines (U) at the 3′ end of the transcript. The target site for the siRNA is conventionally chosen so that the U's at the 3′ end of the anti-sense strand can anneal to one or two A's in the target mRNA. These U residues will be complementary to the A or AA that precede the first G in the sense strand target. Having a complementary adenosine in the target mRNA is probably not essential, however, because hairpins that have the anti-sense strand preceding the sense strand will not have the terminal U's and some hairpins of this orientation are known to be functional.

Currently it is not entirely predictable whether or not a given hairpin siRNA will be effective for RNAi. Each design must be empirically tested [Paddison and Hannon 2002]. Recommended design criteria are similar to those for synthetic siRNA [Elbashir, Harborth et al. 2002]. The target mRNA sequence is chosen to have minimal secondary structure and to be unique (not present in other gene products). Runs of more than 3 bases in the siRNA are not recommended. Hairpin siRNA may include two or three G:U wobble base-pairings in the sense strand of the hairpin in order to stabilize the DNA if it will be propagated in bacteria. The wobble bases are substituted in the sense strand so that effectiveness of the anti-sense strand for RNAi is not compromised. The first base of the stem can be mismatched and it is possible that some other mismatches may be allowed. MicroRNAs can have a few mismatched or bulged bases in the stem of the hairpin and they retain function.

Transfection reagent: A transfection reagent or delivery vehicle is a compound or compounds that bind(s) to or complex(es) with oligonucleotides and polynucleotides and mediates their entry into cells. Examples of transfection reagents include, but are not limited to, cationic liposomes and lipids, polyamines, calcium phosphate precipitates, histone proteins, polyethylenimine, and polylysine complexes. It has been shown that cationic proteins like histones and protamines, or synthetic polymers like polylysine, polyarginine, polyornithine, DEAE dextran, polybrene, and polyethylenimine may be effective intracellular delivery agents. Typically, the transfection reagent has a component with a net positive charge that binds to the oligonucleotide's or polynucleotide's negative charge. The transfection reagent mediates binding of oligonucleotides and polynucleotides to cells via its positive charge (that binds to the cell membrane's negative charge) or via ligands that bind to receptors in the cell. For example, cationic liposomes or polylysine complexes have net positive charges that enable them to bind to DNA or RNA. Polyethylenimine, which facilitates gene transfer without additional treatments, probably disrupts endosomal function itself.

The terms naked nucleic acid and naked polynucleotide indicate that the nucleic acid or polynucleotide is not associated with a transfection reagent or other delivery vehicle that is required for the nucleic acid or polynucleotide to be delivered to the cell.

Delivery: Delivery of a nucleic acid means to transfer a nucleic acid from a container outside a cell to within the outer cell membrane. The term transfection is used herein, in general, as a substitute for the term delivery, or, more specifically, the transfer of a nucleic acid from directly outside a cell membrane to within the cell membrane. If the nucleic acid is a DNA or cDNA, it enters the nucleus where it is transcribed into a RNA.

A delivery system is the means by which a biologically active compound becomes delivered. That is all compounds, including the biologically active compound itself, that are required for delivery and all procedures required for delivery including the form (such as volume and phase (solid, liquid, or gas)) and method of administration (such as but not limited to oral or subcutaneous methods of delivery).

Polynucleotide: The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations of DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a sequence. The term recombinant as used herein refers to a polynucleotide molecule that is comprised of segments of polynucleotide joined together by means of molecular biological techniques. The cassette contains the sequence of interest along with any other sequences that affect expression of the sequence of interest. An expression cassette typically includes a promoter (allowing transcription initiation), and a transcribed sequence. Optionally, the expression cassette may include, but is not limited to, transcriptional enhancers, non-coding sequences, splicing signals, transcription termination signals, and polyadenylation signals.

The polynucleotide may contain sequences that do not serve a specific function in the target cell but are used in the generation of the polynucleotide. Such sequences include, but are not limited to, sequences required for replication or selection of the polynucleotide in a host organism.

A polynucleotide can be used to modify the genomic or extrachromosomal DNA sequences. This can be achieved by delivering a polynucleotide that is expressed. Alternatively, the polynucleotide can effect a change in the DNA or RNA sequence of the target cell. This can be achieved by hybridization, multi-strand polynucleotide formation, homologous recombination, gene conversion, or other yet to be described mechanisms.

Filler DNA is used to expand the size of a plasmid without causing detriment to expression in mammalian cells. The filler DNA sequence used in this invention has a much lower content of the dinucleotide sequence CG (often referred to as CpG) than the average statistical frequency of 1 in 16 that is found in bacterial DNA. The lower CpG content is less likely to activate toll receptors that recognize bacterial DNA. The filler DNA originates from a euchromatin region of mammalian genomic DNA and is expected to reduce the likelihood of the plasmid becoming tightly wrapped into heterochromatin, which could be expected to inactive the expression cassette.

### EXAMPLES

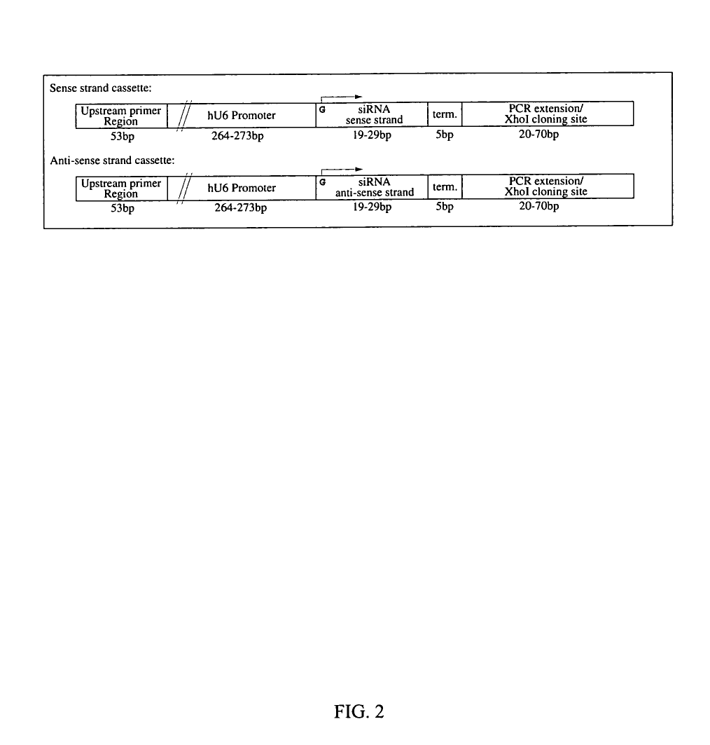

1. Expression cassettes to separately generate sense and anti-sense RNA transcripts. FIG. 2 illustrates components that are part of expression cassettes for separately generating RNA strands to form siRNA within cells. These components include an upstream PCR primer binding site (upstream primer region), a Pol-III promoter (hU6 in this example), a transcription template that starts with G at +1 and codes for either the sense or the anti-sense strand of the siRNA, a (T)5 termination signal, and an extension sequence that may include a restriction site for cloning. An upstream PCR primer binding site is either upstream of or within the promoter. The siRNA strands are 19 to 29 bases long in this example. In the downstream primer, the complement of the termination signal is a run of 5 adenosines. The optional extension sequence at the 5′ end of the downstream primer can be any length and can include a restriction site (Xho I used in this example) for cloning the DNA fragment. For separate strand cassettes, a different target-specific downstream primer is required for each siRNA strand. Each downstream PCR primer has the following general sequence (the underlined base is the site of transcription initiation): 5′-(extension)₀₋₇₀-(A)₅-G-(siRNA sense or anti-sense sequence)₁₉₋₂₉-C-promoter sequence-3′. The promoter sequence of the downstream primer for the human U6 promoter would be SEQ ID 1.

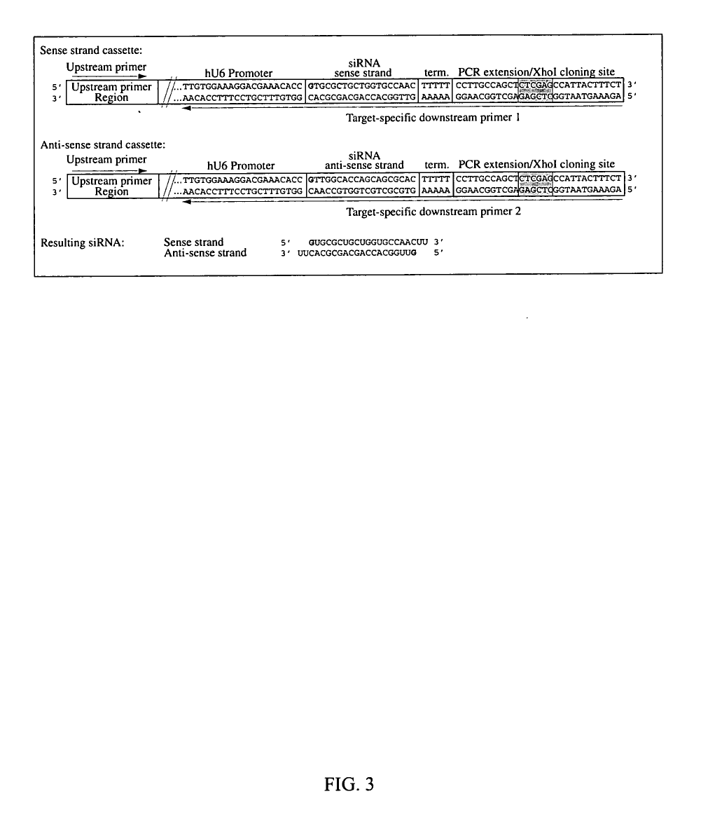

FIG. 3 shows an example of hU6 expression cassettes for separate sense and anti-sense strands targeting firefly luciferase+ (Luc+). These PCR fragments include a downstream extension of 20 bp beyond the transcription termination signal. In this example, the target-specific downstream primer sequences are: Sense strand cassette SEQ ID 3 and Anti-sense strand cassette SEQ ID 4. The C at position 63 of the antisense strand is the complement of the G that codes for the +1 position of the sense RNA transcript. The resulting coding strands in the expression cassettes are SEQ ID 5 and SEQ ID 6, respectively. An Xho I restriction site in the extension sequence is shaded. The transcripts from the two expression cassettes combine in the cell to form the indicated siRNA (sense strand SEQ ID 7 and anti-sense strand SEQ ID 8).

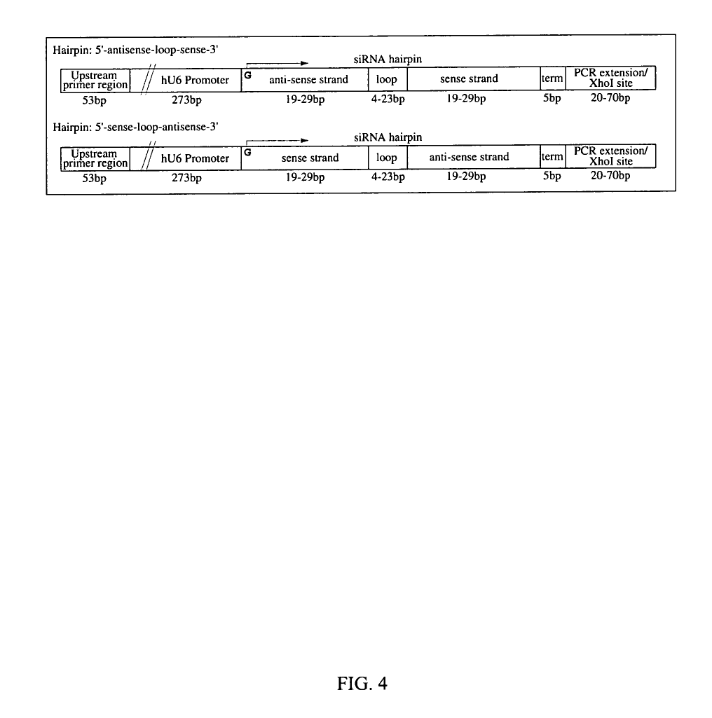

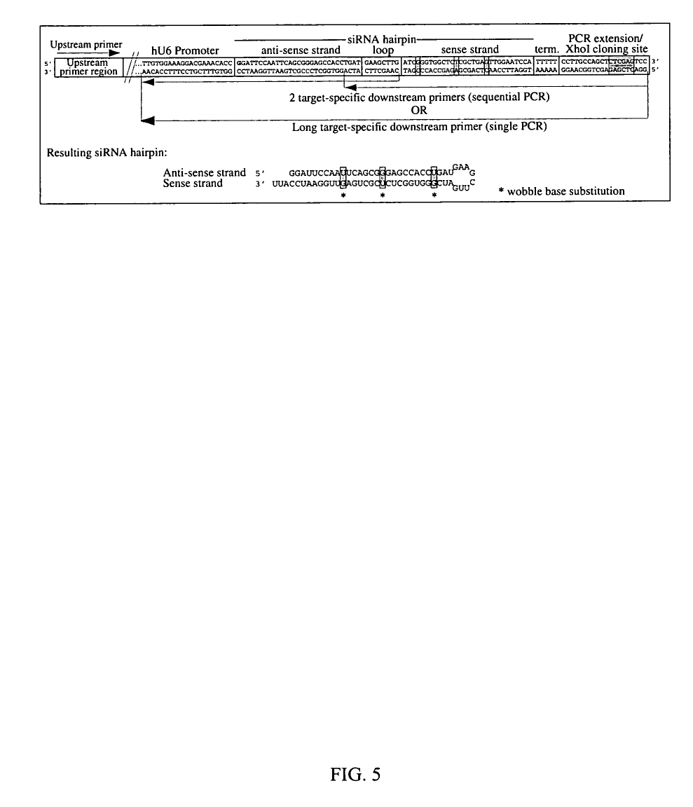

2. Expression cassettes to generate hairpin transcripts. FIG. 4 illustrates expression cassettes for generating short hairpin RNAs. Two designs are shown. The upper design codes for a hairpin that is anti-sense RNA, loop, sense RNA. The lower design codes for a sense, loop, anti-sense hairpin. In the depicted examples, the siRNA expression cassettes encode 19-29 bases of sequence and the complement of that sequence. In both orientations, anti-sense-loop-sense or sense-loop-anti-sense, a guanine is the first transcribed base. The sense and anti-sense strands are separated by 4 to 23 bases that form the loop of the hairpin. Loops can be any sequence. The 5-T termination signal is followed by a 3′ extension sequence that improves efficacy of the cassette. These cassettes have a PCR primer binding site upstream of or within the promoter (upstream primer region). The primer binding sites may include a restriction enzyme site. MicroRNA loop sequences are favored because they may allow more of the nuclear-transcribed hairpin to be exported to the cytoplasm [Kawasaki 2003]. Examples of loop sequences are 5′-CTTCCTGTCA-3′ (SEQ ID 9) from the human mir-23 transcript and 5′-TTCAAGAGA-3′ (SEQ ID 10) from the C. elegans let-7 transcript. The RNA can be in either orientation, anti-sense-loop-sense or sense-loop-anti-sense. Two or three G:U wobble base-pairings may be introduced into the sense strand of the hairpin in order to stabilize the DNA during propagation in bacteria. The wobble bases are substituted in the sense strand so that effectiveness of the anti-sense strand for RNAi is not compromised. FIG. 5 shows an example of a PCR product expression cassette that has the human U6 promoter (hU6) driving a short hairpin transcript that is anti-sense-loop-sense (SEQ ID 12) and targets the luciferase gene at SEQ ID 11. The sequence of the resulting transcript is also shown (SEQ ID 13). Wobble base pairs are shaded and marked with an asterisk. The expression cassette includes the hU6 promoter immediately 5′ of the anti-sense strand sequence of firefly luc+, a loop sequence that includes a Hind III restriction site, the sense strand sequence of luc+ with three wobble base substitutions, a transcription termination signal composed of 5 thymidines, and a downstream extension sequence that includes the Xho I restriction site. This fragment is generated by either two sequential PCR reactions with different downstream primers or by a single PCR reaction with one long downstream primer. A single primer would have the sequence, SEQ ID 14. Both methods utilize the same upstream primer, which anneals to the template DNA upstream of the promoter. The terminology used to describe this sequence is hU6-sh1-luc-X to indicate human U6 promoter, short hairpin design number 1, luciferase+as the target for the siRNA, and an extension sequence that includes the Xho I restriction site downstream of the termination signal.

The target-specific downstream primers for hU6 hairpin cassettes have the following general sequence: where “c” represents the complementary strand and the underlined base is the site of transcription initiation. For an expression cassette to generate a sense-loop-anti-sense hairpin RNA, the downstream primer for a single PCR would be: 5′-(extension)₂₀₋₇₀(A)₅(c-antisense strand)₁₉₋₂₉(c-Loop)₄₋₂₃(c-sense strand)₁₈₋₂₈C(c-promoter sequence)-3′. For an expression cassette to generate an anti-sense-loop-sense hairpin RNA, the downstream primer for a single PCR would be: 5′-(extension)₂₀₋₇₀(A)₅(c-sense strand)₁₉₋₂₉(c-Loop)₄₋₂₃(c-antisense strand)₁₈₋₂₈C(c-promoter sequence)-3′. Because the hairpin cassette target-specific downstream primer can be very long (>100 bases), an alternative for generating hairpin expression cassettes is to design two shorter overlapping target-specific downstream primers (as illustrated in FIG. 5). In this case, the expression cassette is generated through sequential PCRs. In the first reaction, the promoter plus one strand of the hairpin and the loop are amplified. In the second reaction, this first PCR product is extended downstream to add on the second half of the hairpin, the termination signal, and any desired 3 prime extension. For use of two sequential reactions, the precise endpoints of the sequential PCR primers may be chosen based on the following design criteria. During a first PCR using Taq polymerase, the enzyme will add an extra 3′ adenosine (A) to the end of the PCR product. To anticipate this 3′ adenosine (A) addition, the 5′ end of the first downstream primer is placed one base downstream from an adenosine (A) in the complementary strand. Alternatively, the first PCR product can be ‘polished’ with a polymerase with 3′ to 5′ nuclease activity (e.g. Vent, New England Biolabs) before the second PCR. If the hairpin loop contains palindromic sequence (such as from a restriction site), the 3′ end of the resultant second downstream primer may contain a long inverted repeat. Since a 3′ inverted repeat sequence can form a stable hairpin and self-dimer, interference with PCR amplification of the desired expression cassette can result. To reduce this problem, a loop sequence that is not palindromic is used and/or the 3′ end of the second downstream primer is placed at a base that codes for a wobble position in the final hairpin siRNA (see example in FIG. 5), so that the 3′ end is not complementary in primer hairpin and self-dimer structures.

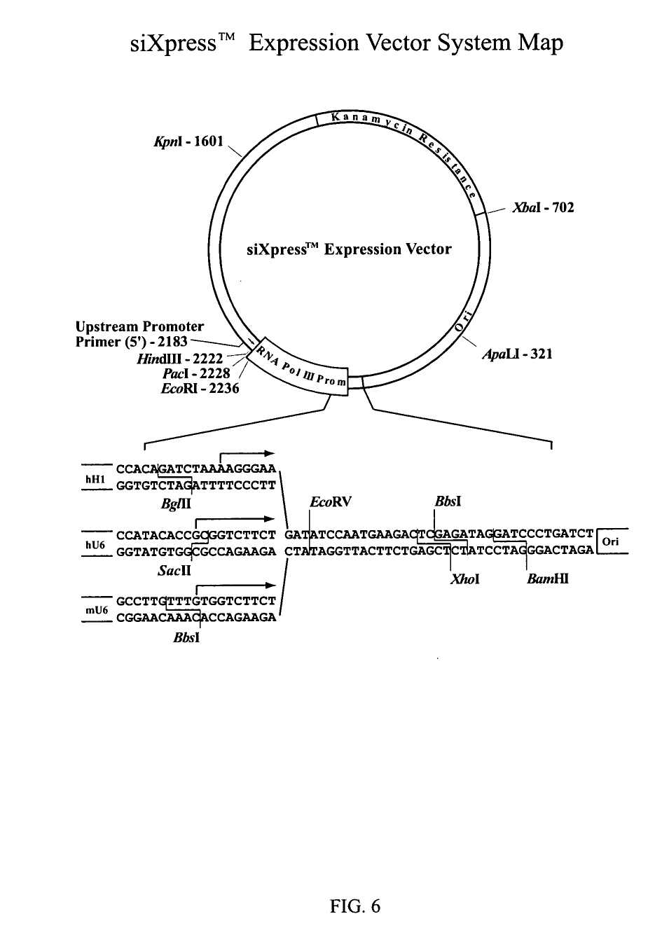

3. Plasmid vectors for siRNA expression. The siXpress™ Expression Vector Systems are kits for expressing siRNA from small fragments of DNA that have either the human H1, human U6 or mouse U6 promoter. The siXpress plasmids are identical from +1 to +2241 and in the multiple cloning sites just upstream of the origin. The vectors include a kanamycin-resistance gene and the bacterial Col E1 origin, and a filler sequence from upstream of the mouse albumin gene. The filler sequence comprises a 686 bp Nhe I/EcoRI fragment from the mouse genomic albumin gene promoter/enhancer region. Unique restriction sites are shown. These plasmids differ only in the promoter. Plasmid pMIR270 has the human H1 promoter, pMIR271 has the human U6 promoter, and pMIR269 has the mouse U6 promoter. The upstream primer for generating siRNA expression cassettes anneals to each of these plasmids 58 bases upstream of the promoter. The sequence of each promoter proximal to the transcription initiation site is shown. The arrows indicate the start site of transcription for each of these promoters. A map of the siXpress plasmid vectors used for the reactions to generate siRNA expression cassettes by PCR is shown in FIG. 6. A blow-up of the sequences in the region of each of these plasmids from upstream of the transcription start site down to the origin is shown.

The upstream primer to amplify a promoter from any of the uniform siXpress™ vectors is PR2-up, SEQ ID 18. To amplify a cassette with a longer sequence upstream of the promoter, primer PR-up, SEQ ID 29, may be used.

4. Generating an siRNA expression cassette by PCR. Expression cassettes generated as in Example 2 with the siXpress plasmids pMIR269, pMIR270 or pMIR271 (SEQ ID 17) described in Example 3 will be referred to as siXpress cassettes. The upstream PCR primer may be PR2-up (SEQ ID 18) or another primer that anneals within or upstream of the promoter (upstream primer region). An example of the hU6-sh1-luc-X siXpress cassette is SEQ ID 20. Cassettes are described by the promoter (e.g., hU6), the RNA type (hairpin, antisense or sense strand) and version of the design (e.g., sh1 indicates short hairpin version #1), the target sequence (e.g., luc indicates luciferase), and the sequence added downstream of the termination signal (e.g., +0 being no added sequence and +25 being 25 bp added). For example, hU6-sh1-luc+25 has the human U6 promoter, short hairpin version 1 targeting luciferase+ and 25 bp downstream of the 5-T termination signal.

Using the method and downstream primers of example 2, the plasmid pMIR271 and upstream primer PR2-up of Example 3, siXpress cassettes were generated. The hU6 promoter drives transcription of a short hairpin targeting luc+ as in FIG. 5. The first PCR product was formed in a 100 μl reaction that was 1×PCR buffer with 0.2 mM of each dATP, dCTP, dGTP and dTTP, 2.5 units ExTaq DNA polymerase (Takara), 50 pmol primer PR2-up (SEQ ID 18), 50 pmol downstream primer hU6/sh1-lucA2 (SEQ ID 15), and 10 ng pMIR271 (SEQ ID 17) as the hU6 promoter template. The cycling conditions were 95° C. for 3 min; 30 cycles of 95° C., 30 sec, 62° C., 20 sec, 68° C., 30 sec; and 68° C. for 10 min. Other cycling conditions could be identified by those skilled in the art. The PCR product from the first reaction was diluted 1:20 with TE (10 mM Tris-HCl, pH 7.9, 0.1 mM EDTA) and 2 μl of the diluted material was used as the DNA template in the second 100 μl reaction. In addition to the first PCR product, the second reaction contained 50 pmol primer PR2-up, 50 pmol primer sh1-lucX (SEQ ID 16), 1×PCR buffer and dNTPs as for the first PCR. The first PCR product was called hU6-sh1-lucA2 and had the sequence (SEQ ID 19). The second PCR product was called hU6-sh1-luc-X had the sequence (SEQ ID 20).

5. siXpress cassettes to target SEAP. Expression cassettes were generated by the siXpress process of Example 4 to target the secreted human placental alkaline phosphatase (SEAP) reporter gene product. Plasmid pMIR271 with hU6 promoter, primer hU6-EcoRI (SEQ ID 25) and downstream primers that encoded either the sense or anti-sense strand to SEAP were utilized. The downstream primers are called versions 3-6. They include 20 bases on the 5′ end of the primer that will allow the amplified cassettes to have 20 bp of sequence downstream of the transcription termination signal. Primers coding for the sense strand of each version are (5′ to 3′): Sequence 3 (SEQ ID 21), Sequence 4: (SEQ ID 22), Sequence 5: (SEQ ID 23), Sequence 6: (SEQ ID 24). These expression cassettes have the hU6 promoter and 20 base downstream extensions. The version 6 sense strand expression cassette is, therefore, called hU6-s6-SEAP+20 and the anti-sense strand cassette is called hU6-a6-SEAP+20. These +20 PCR products were further amplified with the same upstream primer and downstream primer SEAP+20+30X to give them 50 bp downstream extensions that included an Xho I restriction site. The resulting version 6 anti-sense strand cassette was then called hU6-a6-SEAP+50. The siXpress cassette hU6-a6-SEAP+50 was digested with Eco RI and Xho I and ligated into the same sites of vector pMIR271, resulting in pMIR285. Likewise, hU6-s6-SEAP+50 was used to generate pMIR286.

6. PCR products amplified from siRNA expression cassette plasmids. A human U6 short luc+ hairpin expression cassette was formed by the method of Examples 2 and 4 except that hU6 promoter plasmid pMIR219 was used as the DNA template in the PCR, the upstream primer was hU6-EcoRI (SEQ ID 25) and the entire hairpin plus termination signal was encoded in one downstream primer. This upstream primer amplified 273 bp that include the human U6 promoter and it has an EcoRI linker for cloning. In describing a PCR fragment by the primers used, EcoRI will be used to indicate hU6-EcoRI. The resulting PCR product was called hU6-sh1-luc+0. It was cloned into the EcoRI and EcoRV sites of pMIR219 (Example 3) to form pMIR225. Human U6 luc+hairpin cassettes of varying lengths were amplified from pMIR225, using primers that anneal to different sequences upstream or downstream of the expression cassette. Standard PCR procedures were used. The siRNA cassette amplified from pMIR225 with primers hU6-EcoRI and ori3 (referred to as pMIR225, EcoRI/ori3) was 383 bp. It had 38 bp downstream of the 5-thymidine transcription termination signal. The siRNA cassette amplified from pMIR225 with primers hU6-EcoRI and DL7 (pMIR225, EcoRI/DL7) was 422 bp and included 77 bp downstream of the termination signal. The 525 bp PCR product pMIR225, DL8/DL7 had the same expression cassette with an additional 103 bp upstream sequence and 77 bp downstream of terminal T's. PCR product pMIR225, EcoRI/XbaI had no additional upstream sequence but had an additional 720 bp downstream sequence, for a total length of 1,065 bp. Similarly, pMIR225, DL8/XbaI was 1,168 bp overall with an additional 103 bp upstream of the promoter and 720 bp downstream of the terminal T's.

Plasmid pMIR228 was constructed like pMIR225 except that it encodes a 19-base sense luc+ sequence (850-869 of the cDNA) instead of the hairpin. Plasmid pMIR229 is identical to pMIR228 except that it encodes the complementary luc+ anti-sense strand.

Plasmid pMIR230 is identical to pMIR229 except that it codes for the anti-sense strand of SEAP, coding sequence 363-383, instead of luc+₊ pMIR231 is identical to pMIR230 except that it codes for the sense strand of SEAP.

A mouse U6 short luc+ hairpin expression cassette was formed like pMIR225 except that mU6 promoter plasmid pMIR213 (Example 3) was used as the DNA template in the PCR and the upstream primer mU6-HindIII was used. The downstream primer encoded the luc+ hairpin. The resulting PCR product was cloned into the HindIII and EcoRV sites of pMIR213 (Example 3) to form pMIR232.

A human H1 luc+ hairpin expression cassette was generated by PCR with pMIR256 as the hH1 promoter plasmid, hH1-EcoRI as the upstream primer, and with a downstream primer encoding the entire luc+ hairpin and termination signal. The resulting PCR product, hH1-sh1-luc, has the 100 bp hH1 promoter and is 180 bp long. It was cloned into the EcoRI and EcoRV sites of pMIR258 (Example 3) to form pMIR236. The hH1-EcoRI/ori3 PCR product of pMIR236 was 221 bp; the PR2-up/ori3 product was 434 bp; and the PR-up/ori3 product was 478 bp.

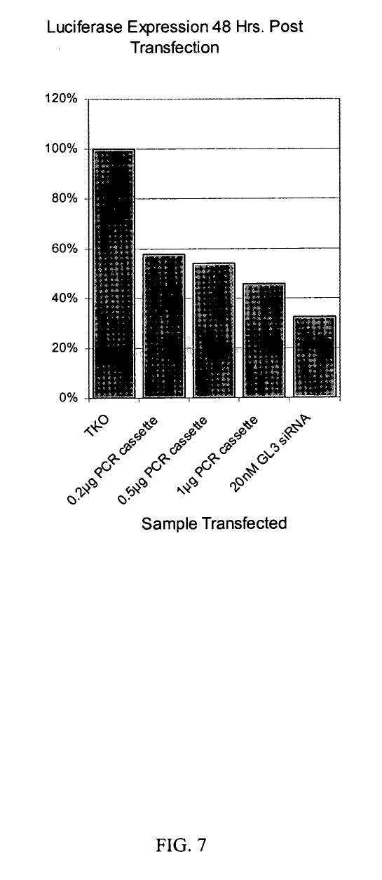

7. Delivery of siRNA expression cassettes to mammalian cell in vitro. A Chinese hamster ovary cell line (CHO) that stably expressed the firefly luc+ gene (CHO-Luc) was plated in 24-well plates with complete media (DMEM, 10% fetal bovine serum, 10 units/ml each streptomycin and penicillin) and transfected at approximately 50% confluency. As in Example 6, luc+ siRNA expression cassettes were amplified from pMIR225 with primers DL8/DL7 to generate a 525 bp product. Cells were transfected with 0.2 μg, 0.5 μg or 1.0 μg PCR product with TransIT-TKO® (TKO) according to the manufacturer's recommendations (Mirus, Madison Wis.). Cells were incubated at 37° C., 5% CO₂ for 48 h. To harvest, wells were rinsed with 1×PBS and then 125 μl LUX lysis buffer (0.1 M KH₂PO₄, pH 7.8, 1 mM DTT, 0.1% Triton X-100) was added. 5 μl of the lysate was assayed for luciferase activity. FIG. 7 shows that luc+ expression was reduced in cells that were transfected with the PCR product luc+ siRNA expression cassettes. A dose of 0.2 μg luc+ siRNA expression cassettes knocked down expression 42%, whereas 1 μg reduced expression 54%. Synthetic siRNA reduced expression 68%.

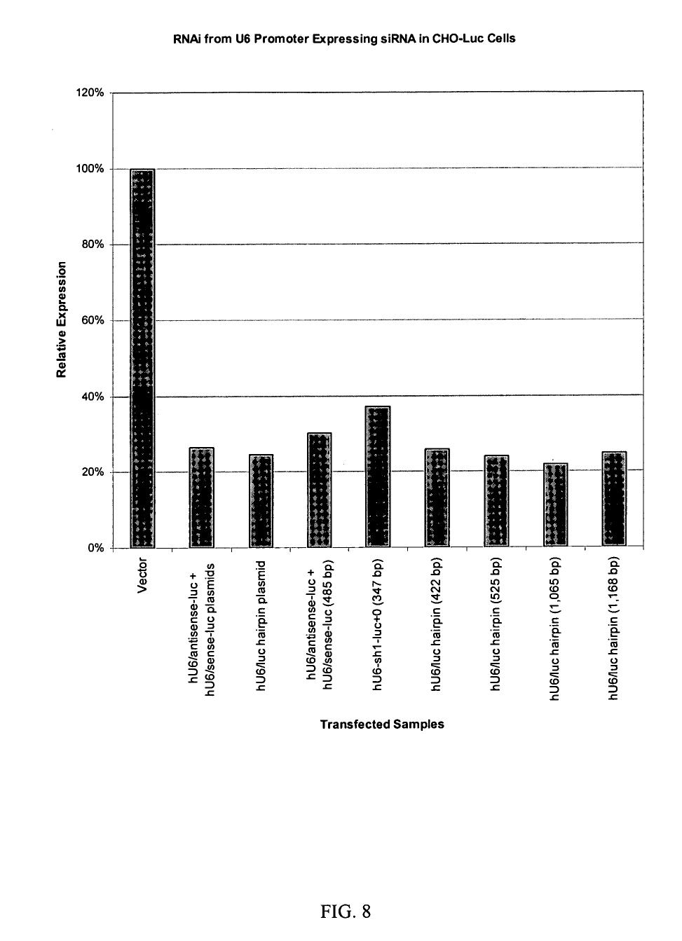

8. Inhibition of gene expression by delivery of hairpin and separate strand siRNA expression cassettes. CHO-Luc cells in 24-well plates were transfected in triplicate at 40% confluency with TransIT-LT1 (LT1) according to the manufacturer's recommendations (Mirus, Madison Wis.). Cells were harvested and assayed for luciferase activity. The transfected DNA samples (described in Example 6) were pMIR35 (Vector); pMIR228/229 (hU6/antisense-luc+hU6/sense-luc plasmids); pMIR225 (hU6/luc hairpin plasmid); pMIR228/229 PCR products (the 485 bp hU6/antisense-luc+hU6/sense-luc fragments); the hU6 luc+ short hairpin expression cassette derived without subeloning that has no extra bases downstream of the termination signal (347 bp hU6-sh1-luc+0); and pMIR225 PCR products from primers hU6-EcoRI/DL7 (422 bp), DL8/DL7 (525 bp), hU6-EcoRI/XbaI (1065 bp) and DL8/XbaI (1168 bp). Results are shown in FIG. 8. Expression was scaled to the empty vector sample. Luciferase expression was knocked down equally well by PCR products from pMIR225 as from the plasmid pMIR225. Separate strand siRNA plasmids pMIR228/229 were as effective as hairpin plasmid pMIR225. PCR products of pMIR228/229 were nearly as effective as the parent plasmids. The hairpin expression cassette with no bases downstream of the termination signal (hU6-sh1-luc+0) was less effective for mediating RNAi than the hairpin expression cassette from pMIR225 that had an extra 77 bp downstream of the termination signal.

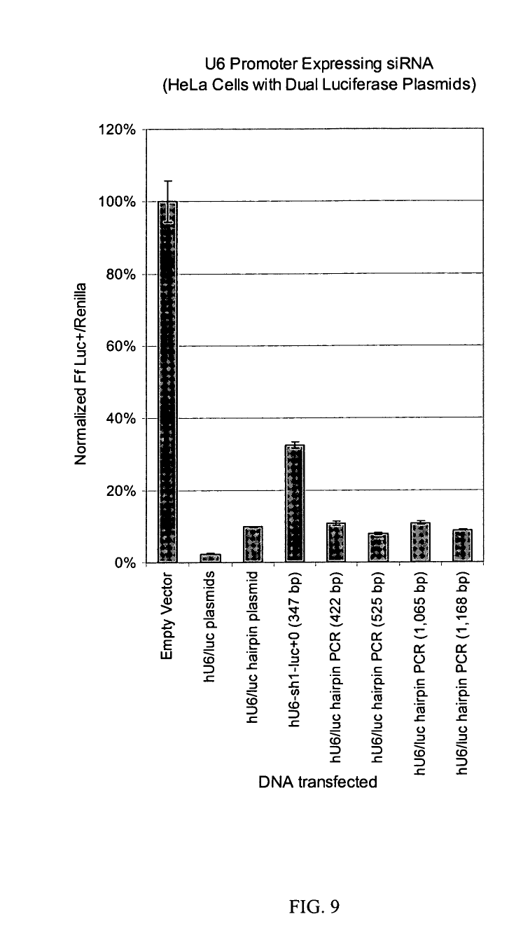

9. Inhibition of gene expression by induction of RNAi following delivery of siRNA expression cassettes. HeLa cells were transfected with the Dual Luciferase reporter plasmids to express firefly luc+ (pGL3-Control) and Renilla luc (pRL-SV40; Promega, Madison Wis.). Each well received a total of 500 ng DNA: 249 ng pGL3-Control, 1.25 ng pRL-SV40 and 250 ng of samples described in Example 6 or the empty vector control pMIR35. Co-delivered with these reporter plasmids were pMIR35 (Empty Vector); pMIR228/229 (hU6/luc plasmids); pMIR225 (hU6/luc hairpin plasmid); the hU6-sh1-luc+0 expression fragment; or pMIR225 PCR products from primers hU6-EcoRI/DL7 (422 bp), DL8/DL7 (525 bp), hU6-EcoRI/XbaI (1065 bp) and DL8/XbaI (1168 bp). Plasmid pRL-SV40 expresses Renilla luciferase and serves as a delivery control in each well of cells. Firefly luc+ is expressed from pGL3-Control and is the target for the siRNA produced by plasmids and PCR products. Cells were lysed and expression of firefly luc+ and Renilla luciferase were measured with the Dual Luciferase Assay (Promega) according to the manufacturer's instructions. For each sample, the ratio of relative light units (RLU) from firefly luc+ to RLU from Renilla luciferase is normalized to this ratio from the samples transfected with the empty vector pMIR35. Normalized expression is presented in FIG. 9. Effective knockdown of luc+ (90%) is mediated by PCR-generated fragments of pMIR225 luc+ hairpin cassettes that range in size from 422 bp to 1168 bp and include 77 bp or more of sequence downstream of the termination signal. In contrast, knockdown from the expression cassette with no additional downstream extension (hU6-sh1-luc+0) was 60%.

10. Small amounts of siRNA expression cassette fragments induce inhibition of gene expression by induction of RNAi. In order to determine if small amounts of siRNA expression cassettes are effective for knockdown of an endogenous gene, a dose of 10 ng, 50 ng, 100 ng, 250 ng or 500 ng of the luc+ hairpin expression cassette hU6+30-sh1-luc+40 (414 bp) was transfected into CHO-Luc, as in Example 8. The hU6+30-sh1-luc+40 expression cassette was generated as in Example 6. The upstream primer hU6-up+30 (SEQ ID 26) had the same sequence as hU6-EcoRI (SEQ ID 25) with an added 30 bases at the 5′ end. The downstream primer in the first PCR encoded the entire luc+ short hairpin. The downstream primer to extend the first PCR product added a 40 bp 3′ extension downstream of the termination signal. The resulting PCR expression cassettes, hU6+30-sh1-luc+40, were cotransfected into cells with non-specific DNA. Non-specific DNA was used to bring the total mass of transfected DNA to 500 ng. As controls, cells were transfected with 500 ng pMIR225 (the positive control luc+ hairpin plasmid), 500 ng pMIR230/231 PCR products expressing siRNA to SEAP (negative control) and a 383 bp luc+ hairpin expression cassette amplified from pMIR225 (positive control). Cells were harvested and assayed for luciferase expression as in Example 7.

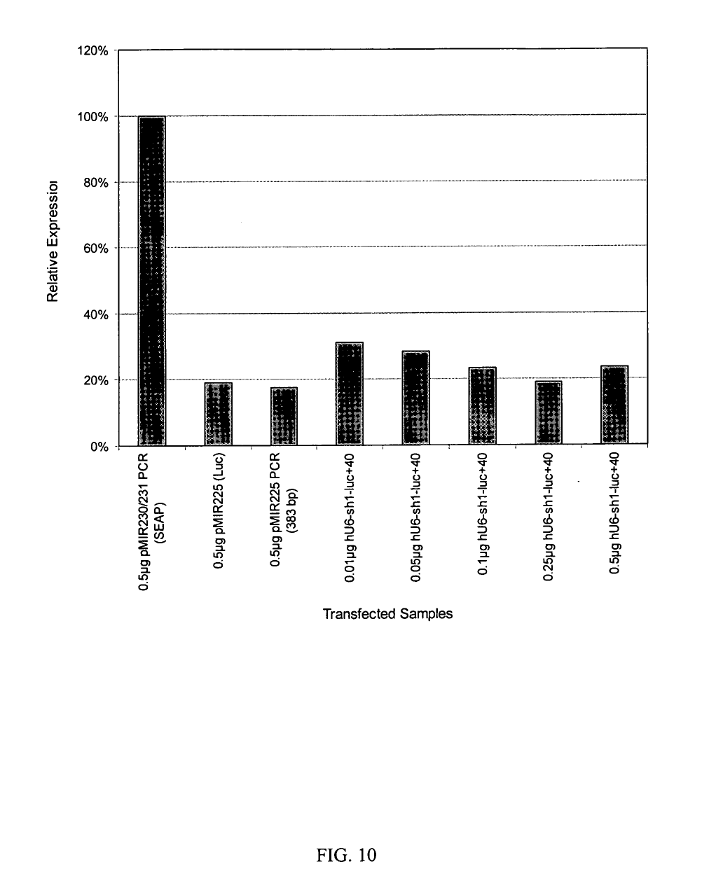

Luciferase expression in samples transfected with fragments of DNA was scaled to expression in cells that received pMIR230/231 PCR (SEAP). Luciferase expression was knocked down 69% in cells that were transfected with only 10 ng of PCR-generated luc+ hairpin expression cassette hU6+30-sh1-luc+40 and 76% in cells that received 500 ng of the cassette (FIG. 10). This expression cassette, generated without subcloning, was efficient for inducing RNAi.

11. Addition of 3′ extensions downstream of the termination signal improves siRNA expression cassette function. As shown in FIG. 9, the hU6 luciferase hairpin cassette with no 3′ extension is less effective for RNAi than the cassette with a 77 bp 3′ extension (pMIR225, EcoRI/DL7). The pMIR225 PCR (383 bp) product generated by hU6-EcoRI primer and ori3 primer has a 38 bp 3′ extension downstream of the termination signal and functioned as well as the plasmid (see FIG. 10).

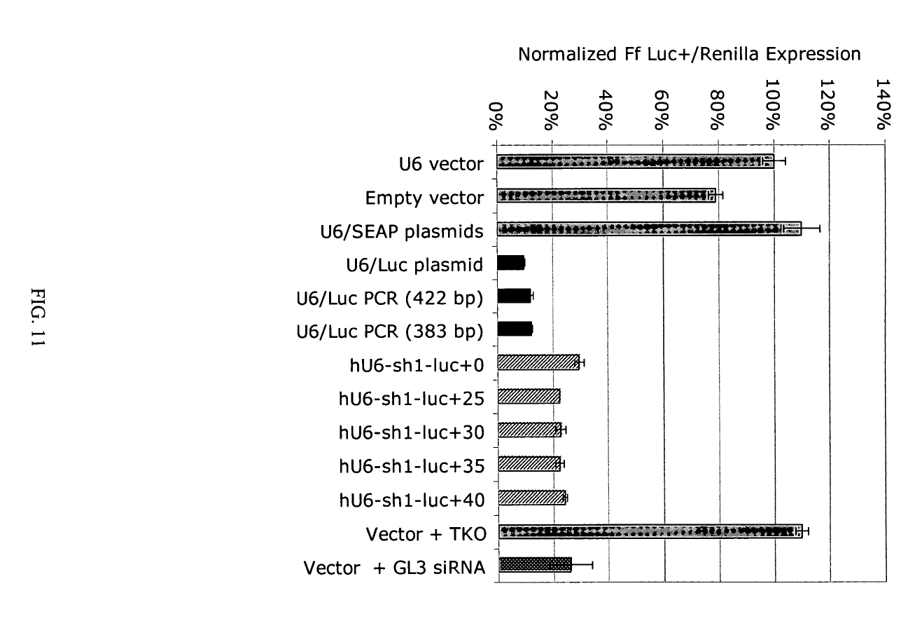

A U6/Luc PCR expression cassette containing a 38 bp 3′ extension (U6/Luc PCR (383 bp)) was as effective as a U6/Luc PCR expression cassette containing a 77 bp 3′ extension (U6/Luc PCR (422 bp), FIG. 11). Expression cassettes generated by PCR, without subcloning, which contained 25 to 40 bp 3′ extension. Expression cassettes with 3′ extensions of 25-40 bp were generated using a sequential PCR approach. A cassette without a 3′ extension, hU6-sh1-luc+0, was generated in the first PCR amplification as described in example 6. This cassette was then used as template in the second round of PCR in which the downstream primers annealed to the downstream half of the hairpin sequence and 5 T's of the first round template but additionally included 25, 30, 35 or 40 bases on the 5′ end of the primer, thus generating 25, 30, 35 or 40 bp 3′ extensions.

HeLa cells in were transfected at 65% confluency with 250 ng of the combined Dual Luciferase plasmids and 250 ng of experimental sample DNA by the procedure described in Example 8. Transfected expression vectors were: pMIR219 (U6 vector); pMIR35 (Empty Vector); pMIR230/231 (U6/SEAP plasmids); pMIR225 (hU6/Luc plasmid); pMIR225 PCR products from primers hU6-EcoRI/DL7 (U6/Luc PCR, 422 bp) or from primers hU6-EcoRI/ori3 (U6/Luc PCR, 383 bp); or hairpin luc+ expression cassettes generated by PCR with 0 (hU6-sh1-luc+0), 25 (hU6-sh1-luc+25), 30 (hU6-sh1-luc+30), 35 (hU6-sh1-luc+35), or 40 (hU6-sh1-luc+40) bases downstream of the termination signal. To compare synthetic siRNA to expression cassettes, control samples were additionally transfected with siRNA targeting luciferase (GL3 siRNA). Cells were harvested, Dual Luciferase assays performed, and samples analyzed by comparison of firefly luc+/Renilla luciferase ratios as in Example 8. All samples were normalized to the U6 vector sample, which was set as 100% and results are presented in FIG. 11.

Firefly expression was inhibited 90% by pMIR225 and 88% by either the 383 or 422 bp pMIR225 PCR product. In this experiment, PCR products generated without subcloning were also effective in inhibiting firefly luciferase expression. Downstream extensions of 25 to 40 bp were all equally effective for mediating RNAi. The PCR generated expression cassettes knocked down firefly luciferase expression as well as the synthetic GL3 siRNA.

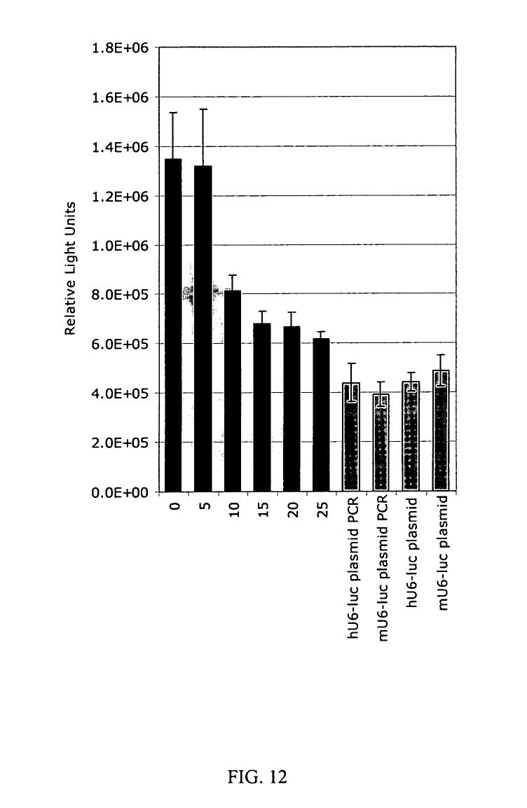

12.3° extensions improve efficacy of small RNA expression cassettes. The hU6-sh1-luc+0 hairpin expression cassette was generated by PCR as in Example 6. The hU6-sh1-luc+0 hairpin expression cassette was extended by secondary PCR amplification with downstream primers that added 5, 10, 15, 20 or 25 bp 3′ extensions downstream of the transcription termination signal using the method described in Example 11. The resulting expression cassettes were transfected into CHO-Luc cells. Cells were incubated for 50 h, then harvested and assayed for luciferase expression as in Example 7. As positive controls, mU6 luciferase hairpin plasmid pMIR232 and a PCR product from this plasmid, hU6 luciferase hairpin plasmid pMIR225 and the 383 bp PCR product from pMIR225 were included. The results are shown in FIG. 12. The hU6-sh1-luc expression cassettes with no added bases or 5 added bases showed little knock down of luciferase expression. Cassettes with 15-25 base 3′ extensions were more effective for mediating RNAi.

13. Analysis of PCR expression cassettes with varying overall and 3′ extension length. Separate hU6 sense strand and anti-sense strand luc+ RNA expression cassettes were cloned into plasmids. hU6-antisense luc (hU6-aB1-luc) and hU6-sense luc (hU6-sB1-luc) PCR expression cassettes, varying in length from 263-457 bp, were amplified from these plasmids. Both the upstream and downstream lengths were varied. The fragment generated by upstream primer hU6-oct included 239 bp upstream of the transcription start site; hU6-EcoRI included 274 bp upstream of the transcription start site; PR2-up included 326 bp upstream of the transcription start site; and PR-up included 371 bp upstream of the transcription start site. Downstream primers added 0 base (hU6/aB1-luc and hU6/sB1-luc), 20 base (Ori+20), 40 base (Ori+40), or 62 base (Ori4) 3′ extension. The control fragments expressed sense and anti-sense strands of SEAP and were generated by PCR of pMIR285 and pMIR286 (see Example 5) and contained varying sequence lengths both upstream and downstream of the SEAP siRNA coding sequence.

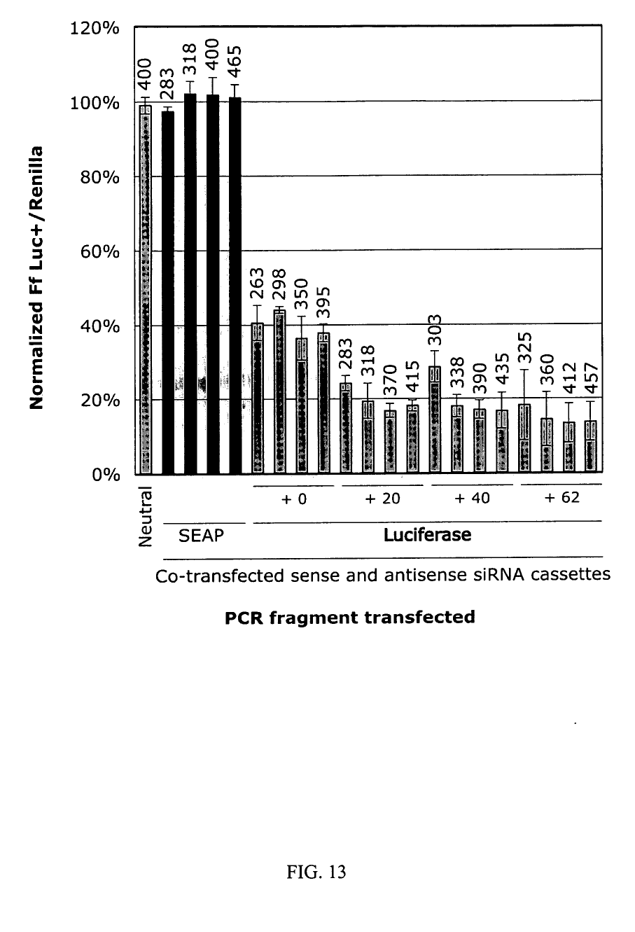

HeLa cells were transfected with LT1 and a total of 500 ng DNA as in Example 8. Each well received 250 ng of combined firefly luc+ plasmid pGL3-Control plus Renilla luciferase plasmid pRL-SV40 and 250 ng (125 ng each sense strand and anti-sense strand expression cassette). The 400 bp PCR fragment of genomic sequence is referred to as Neutral. Cells were harvested 48 h after transfection and assayed for firefly and Renilla luciferase as in Example 9. Results are shown in FIG. 13. The ratio of firefly to Renilla luciferase was essentially the same between all wells transfected with the Neutral fragment or with any of the SEAP expression cassettes. The average luciferase expression from the 4 SEAP samples was set at 100%.

The luciferase siRNA cassettes with no extensions (+0) all mediated approximately 60% knockdown, regardless of upstream length. 3′ extensions of 20-62 bp were similarly effective for mediating RNAi, resulting in greater than 80% knockdown on average. There was little to no effect on efficacy between expression cassettes with different upstream lengths but identical 3′ extensions. Thus, the presence of a 3′ extension of sufficient length is important for the effectiveness of the expression cassette. The 3′ extension enhances the effectiveness of both hairpin cassettes and separate strand cassettes. The preferred 3′ extension length is similar for both hairpin and separate strand expression cassettes even though the overall size of the cassettes and the distance from transcription start to the downstream end of the fragments differ.

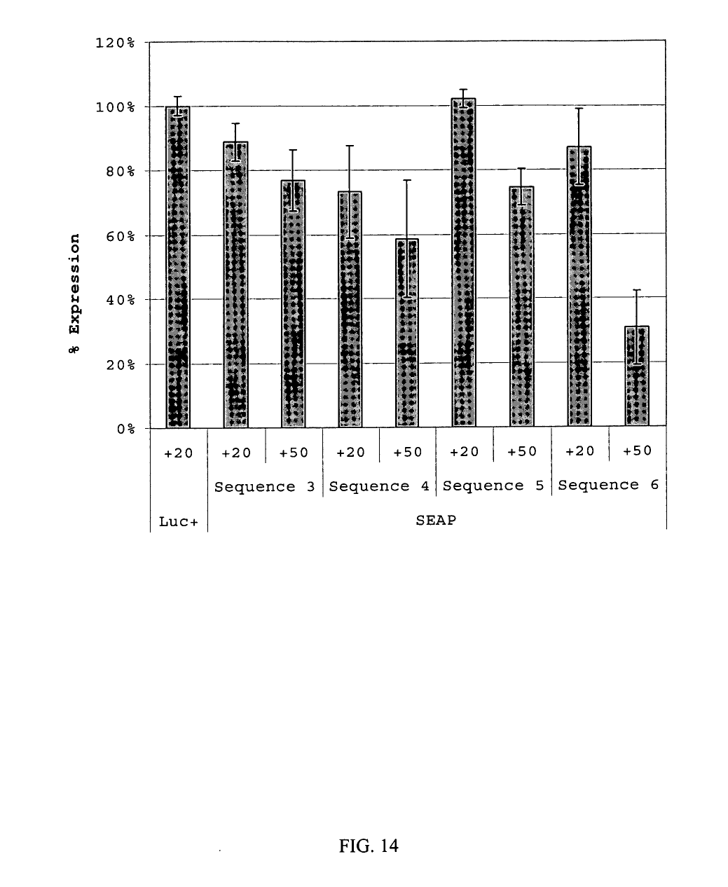

14. Using PCR generated siRNA expression cassettes to screen for effective siRNAs. In order to find an effective siRNA sequence to target SEAP, several potential siRNA sequences were tested using separate strand siRNA expression cassettes. SEAP cassette versions 3-6, consisting of different sequences derived from the SEAP gene, are described in Example 5. For each version, siXpress cassettes were generated by PCR with 20 or 50 nucleotide 3′ extensions (+20 or +50) downstream of the termination signal. These cassettes were tested in the mouse liver cell line BNL CL.2. The BNL cells were plated at 30,000 cells per well in complete medium (DMEM, 10% fetal bovine serum, 10 units/ml Pen/Strep) in 24-well plates. The next day each well was cotransfected with the sense and anti-sense cassettes using 1.5 μl LT1 according to the manufacturer's recommendations. Cells were incubated at 37° C., 5% CO₂, for 72 h. Then the medium was replaced with fresh medium. After 3.5 h, 100 μl of the medium was drawn from each well and assayed for SEAP activity using the Tropix Phospha-Light™ chemiluminescent reporter assay kit, according to the manufacturer's instructions. A hU6 luciferase expression cassette was used as a control. Relative light units of SEAP activity from the cells that were transfected with the luc cassette was set to 100%. Results are shown in FIG. 14.

For each SEAP expression cassette design, the +50 3′ extension fragment was more effective for knockdown than the +20 3′ extension. The most effective siRNA was Version 6. The ease of producing the expression cassettes and there effectiveness is generating functional siRNA thus readily enables one to screen different sequences to find an optimal siRNA sequence.

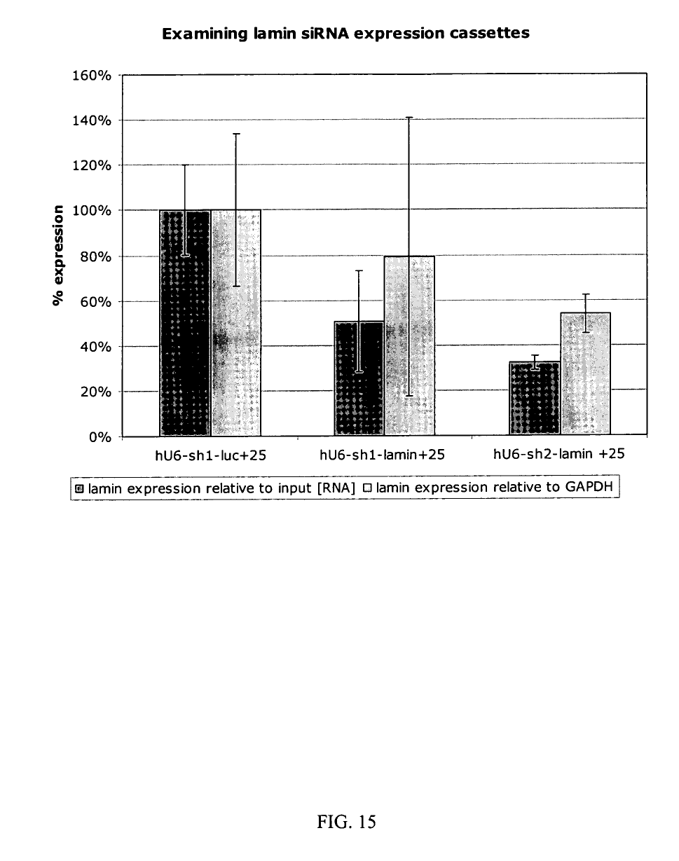

15. PCR generated siRNA expression cassettes are effective in inhibiting and endogenous gene. Hairpin siRNA expression cassettes were designed to target mouse lamin A/C. Expression cassette hU6-sh1-lamin was generated from plasmid pMIR219 with upstream primer hU6-EcoRI and downstream primer hU6/shRNA1-lamin (SEQ ID 27) as in Example 4. The resulting PCR product was hU6-sh1-lamin. It was extended by a second amplification with the same upstream primer and downstream primer sh1-lamin+25 that added a 25 bp downstream extension. Cassette hU6-sh2-lamin+25 was generated the same way except with downstream primer hU6/shRNA2-lamin (SEQ ID 28) in the first PCR and downstream primer sh2-lamin+25 in the second PCR. These PCR products were transfected into BNL cells in 12-well plates with 1.5 μg LT1 and 500 ng DNA fragments per well. The luciferase hairpin cassette hU6-sh1-luc+25 was used as a control. Cells were incubated at 37° C. for 72 h and the mRNA levels for lamin A/C were evaluated by Q-PCR [Zheng 2001]. The hU6-sh2-lamin+25 knocked down expression of endogenous lamin A/C by approximately 70% (FIG. 15).

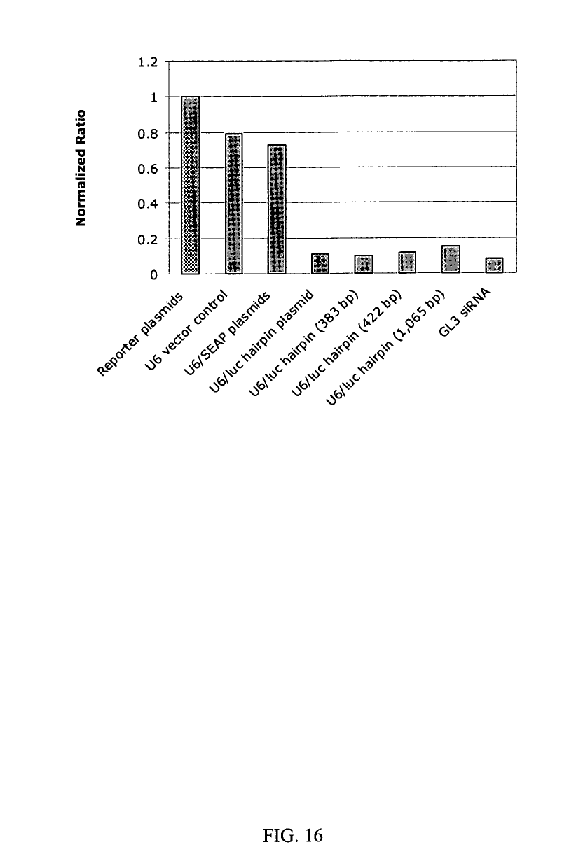

16. Small siRNA expression cassettes are effective in inhibiting gene expression in vivo. In order to determine if the describe expression cassettes could mediate RNAi in vivo, a Dual Luciferase approach was used to control for DNA delivery, similar to the procedure used by Lewis et al. [Lewis 2002]. ICR mice (n=4) were co-injected via tail vein injection [Zhang 1999] with two reporter plasmids, firefly luc+ (pGL3-Control) and Renilla luciferase (pRL-SV40). Each mouse was injected with 15 μg pGL3-Control and 0.1 μg pRL-SV40. Co-delivered with the reporter plasmids were 10 μg of either hU6 vector plasmid pMIR219, combined SEAP siRNA plasmids pMIR230/231, pMIR225 plasmid-base firefly luc+ siRNA expression cassette, or PCR fragments from the pMIR225. The luciferase specific-siRNA PCR expression cassettes were the 383 bp EcoRI/ori3; the 422 bp pMIR225, EcoRI/DL7; and the 1065 bp pMIR225, EcoRI/XbaI (see Example 6). Synthetic siRNA to target luc+ (GL3 siRNA) was co-delivered with reporter plasmid in one group. Mice were sacrificed 54 h after injection. The livers were harvested and homogenized in LUX lysis buffer (Example 7). The Dual Luciferase assays were performed as in Example 9. The averaged ratios of firefly/Renilla expression in each group of mice was normalized to the ratio from the mice that were injected with reporter plasmids only. Results are shown in FIG. 16. Firefly luciferase was inhibited 89% from plasmid pMIR225, 90% from the 383 bp pMIR225 fragment, 88% from the 422 bp pMIR225 fragment, 85% from the 1065 bp pMIR225 fragment, and 92% from the synthetic GL3 siRNA. The PCR fragments mediated RNAi in the mice to the same degree as the siRNA expression plasmid and the synthetic siRNA indicated their utility in vivo.

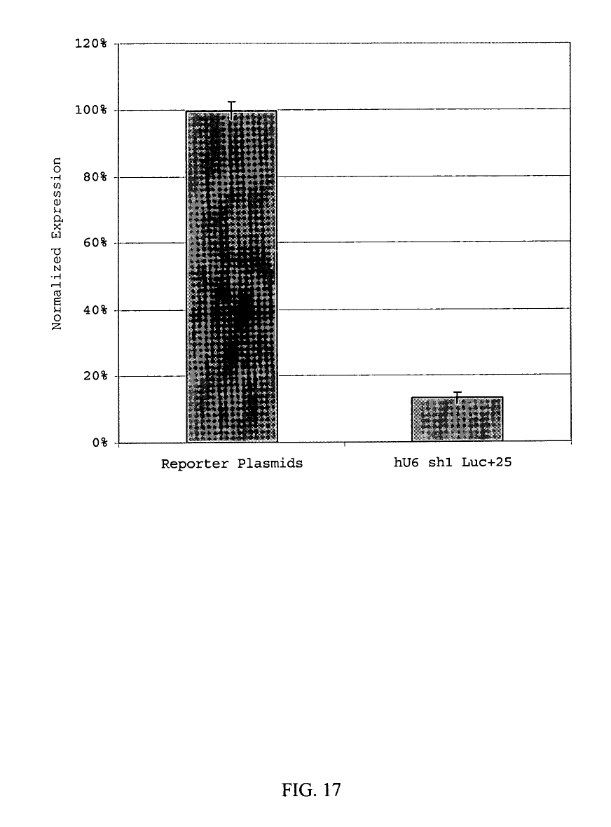

17. PCR-based siRNA expression cassette in vivo (without sub-cloning). The hU6-sh1-luc+25 expression cassette was generated as in Example 4 from two sequential reactions. The first reaction included the hU6 promoter vector pMIR219, the upstream primer hU6-EcoRI, and a downstream primer that encoded the entire luc hairpin and termination signal. The product of this first reaction was hU6-sh1-luc+0 as in Example 6. A 25 base 3′ extension was added end by a second amplification using the first product as template, the same upstream primer, and sh1-luc+25 as the downstream primer. The resulting expression cassette was evaluated by co-delivery with firefly luciferase plasmid pMIR68 and the Renilla luciferase reporter plasmid pRL-SV40. Plasmid pMIR68 expresses a much higher level of firefly luciferase after 2 days than the CMV plasmid pGL3-Control does, so that expression can be evaluated over several days. C57BL/6 mice were injected as in example 16 with 10 μg pMIR68 plus 10 μg pGL3-Control. One group of mice was injected with 10 μg hU6-sh1-luc+25 expression cassettes together with the reporter plasmids. After 3 days, the livers were harvested, homogenized and assayed for firefly and Renilla luciferase as in Example 16. The firefly/Renilla ratios of mice that received the expression cassettes were normalized to the mean ratio of mice receiving reporter plasmids only. The small fragments of DNA that expressed short hairpin RNA, and were generated by PCR without sub-cloning, specifically knocked down firefly luciferase by 87% (FIG. 17).

18. Human H1 promoter driving siRNA from small fragments of DNA in vivo. The hH1 promoter (100 bp) is much smaller than the hU6 promoter (264 bp). We sought to determine if a small fragment of DNA bearing the hH1 promoter in an siRNA expression cassette could mediate knockdown in vivo. ICR mice were injected in the tail vein with 1 μg firefly expression plasmid pMIR68 and 20 μg Renilla luciferase expression plasmid pMIR252. Co-delivered with the reporter plasmid were 10 μg of either pMIR263 plasmid or pMIR263 PCR products from primers hH1-EcoRI/ori3 (221 bp), PR2-up/ori3 (434 bp), or PR-up/ori3 (478 bp), described in Example 6. On day 3 the mice were sacrificed and livers were harvested, homogenized, and assayed for Dual Luciferase activity as in Example 16. The firefly/Renilla ratios were normalized to reporter plasmids only. Firefly luciferase expression was knocked down 42% by plasmid pMIR263, 50% by the 221 bp fragment, 56% by the 434 bp fragment, and 32% by the 478 bp fragment of pMIR263.

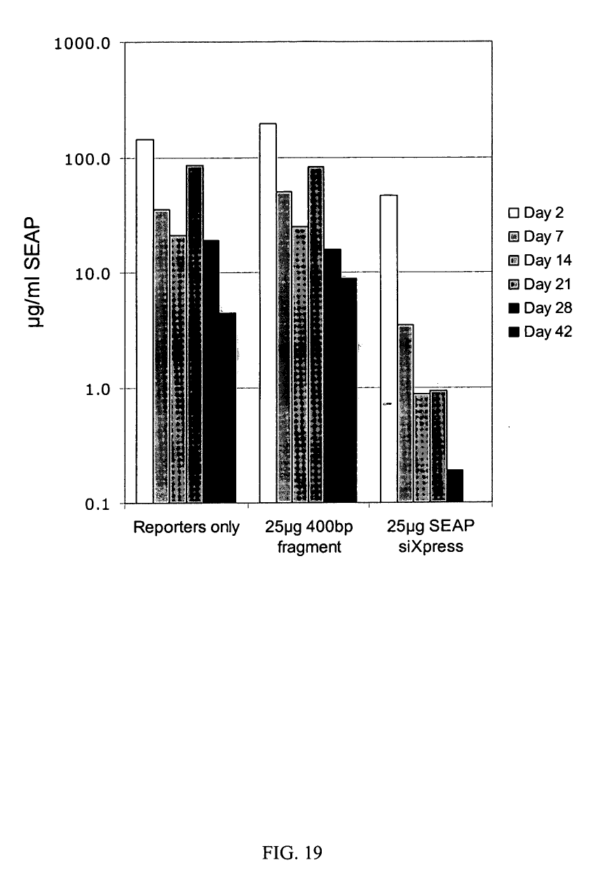

19. Small fragments of DNA mediate long-term RNAi. SEAP siXpress cassettes were screened for activity by co-delivery with a SEAP reporter gene plasmid in cultured cells (Example 14). The pair of sense and anti-sense expression cassettes that showed the greatest target gene knock-down were cloned into the siXpress vector pMIR271 (SEQ ID 17) to generate pMIR286 and pMIR285 (Example 5). Fragments of 400 bp were amplified from pMIR285 and pMIR286 with primers PR2-up and SEAP+20+30. These separate strand expression cassettes included 50 bp extensions downstream of the termination signal. The pMIR285/pMIR286 PCR products (25 μg) were co-delivered with 1.0 μg pMIR141 SEAP reporter plasmid and 20 μg pMIR254 delivery control plasmid into C57BL/6 mice by tail vein injection (n=4). For long-term evaluation of siRNA knock-down in vivo mediated by expression cassettes, the SEAP reporter plasmid pMIR141 was used. SEAP is stably expressed from pMIR141 in C57BL/6 mice at very high levels for over one year. The control plasmid pMIR254 expressing human alpha-1 anti-trypsin. As controls, one group of mice received reporter plasmids only and another received reporter plasmids plus 25 μg of a 400 bp fragment of genomic sequence (400 bp Neutral). Blood was drawn from the mice by retro-orbital bleeding to collect serum at 2, 7, 14, 21, 28, and 42 days post injection. The amount of SEAP in the serum was measured with the Tropix chemiluminescent assay as in Example 14. FIG. 19 shows the average amounts of SEAP produced in each group of mice. SEAP expression in the group of mice that received SEAP siRNA cassettes is compared to SEAP expression in the group that received an equal amount of DNA of the same size (the 400 bp Neutral group). At 2 days post injection, SEAP expression in the group that received the pMIR285/pMIR286 PCR products was reduced 76%. At day 7 the knock-down was 93%, and at day 14 it was 99%. In summary, the siXpress cassettes that functioned best in vitro were highly efficient for mediating RNAi in vivo.

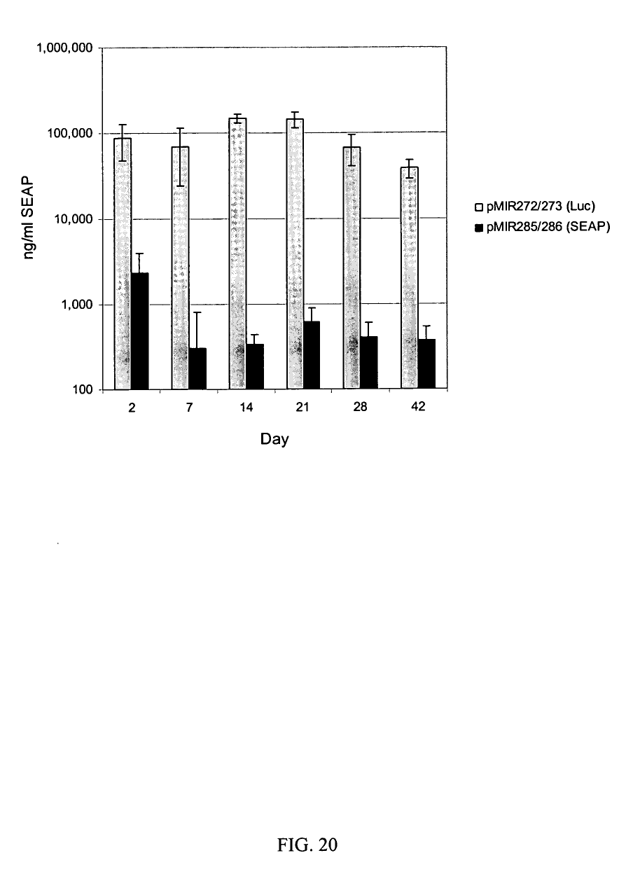

20. An expression vector for long-term expression of siRNA. The version 6 separate sense and anti-sense strand SEAP siRNA expression cassettes were sub-cloned into plasmid vector pMIR271 (SEQ ID 17) to generate plasmids pMIR285 and pMIR286 (Example 5). C57BL/6 mice were injected in the tail vein with 1 μg SEAP reporter plasmid pMIR141, 10 μg delivery control plasmid pMIR174 for human factor IX expression, and 50 μg of either pMIR272/pMIR273 expressing separate strand siRNA targeting luc+ or pMIR285/pMIR286 siRNA plasmids targeting SEAP. Serum was collected from the mice at 2, 7, 14, 21, 28 and 42 days post injection. SEAP in the serum was detected as in Example 14. FIG. 20 shows SEAP reporter gene expression in mice that were injected with the luciferase control siRNA plasmids (pMIR272/273) versus the SEAP siRNA plasmids (pMIR285/286). The SEAP reporter gene expression was knocked down 38-fold at day 2 post injection and over 400-fold at day 14. RNAi endured from these vectors for at least 42 days (FIG. 20).

21. RNA polymerase II expression cassettes for siRNA. Expression cassettes are generated by PCR from a transcription control unit that encodes a promoter or an enhancer plus a promoter. The transcription control unit can be encoded in a plasmid. The upstream primer is upstream of this transcription control unit. The siRNA sequence is encoded in the downstream primer similar to Examples 1 and 2. The downstream primer for Pol-II does not need an extension sequence downstream of the siRNA. It may have 2 to 5 A's at the 5′ end of the primer, but the Pol-II promoter does not use a 5-T termination signal. The +1 position of the promoter is maintained and the siRNA sequence follows. For example, the downstream end of the mouse albumin promoter and first base of the endogenous albumin transcript (underlined) are 5′- . . . AGAGCGAGTCTTTCTGCACACA-3′ (SEQ ID 30). The downstream primer has the following general sequence (the underlined base is the site of transcription initiation and the complement of the promoter sequence is used): 5′-A₂₋₅ ( . . . complement of siRNA strand . . . T)₁₉₋₂₉ GTGTGCAGAAAGACTCGCTCT-3′ (SEQ ID 31). The PCR reaction is carried out as in Example 4 or with similar amplification conditions known to those skilled in the art. In the case of a hairpin primer, the entire hairpin can be encoded in the downstream primer. Alternatively, two sequential PCR reactions would be used as is done to generate the Pol-III cassettes. The first reaction primer to generate a hairpin that is anti-sense strand, loop, sense strand would have the general sequence (where “c” indicates complement of and the underlined base is the transcription start site): 5′-(c-loop-c-anti-sense siRNA strand . . . T) c-promoter-3′. The second primer would have the general sequence: 5′-A₂₋₅ (c-sense siRNA strand-c-loop). The primer sequences are designed to accommodate the adenosine added by Taq DNA polymerase as described in Example 2. In summary, a single PCR reaction generates a sense or anti-sense strand siRNA cassette with a Pol-II promoter. The hairpin siRNA cassette is generated by one or two PCR reactions.

Examples of enhancer/promoter combinations that give high levels of sustained expression in liver are the minimal alpha-fetoprotein (AFP) enhancer elements with the albumin promoter. The mouse AFP enhancer element I is 300 bp and the mouse AFP enhancer element II is 220 bp. The mouse albumin promoter from −281 to +1 is an example of a Pol-II promoter that can be used for liver-specific expression cassettes. Other liver-specific enhancers or promoters can be utilized. For tissue-specific expression in other tissues, enhancer/promoter combinations that are specific for those target tissue can be used.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A process for delivering a small RNA to a cell comprising: forming a linear expression cassette and delivering the cassette to a cell wherein the small RNA is expressed.

2. The process of claim 1 wherein the expression cassette consists of a linear DNA fragment.

3. The process of claim 2 wherein the expression cassette contains an RNA polymerase III promoter.

4. The process of claim 3 wherein the RNA polymerase III promoter consists of the U6 promoter.

5. The process of claim 3 wherein the RNA polymerase III promoter consists of the H1 promoter.

6. The process of claim 2 wherein forming a linear expression cassette comprises PCR amplification of a template.

7. The process of claim 6 wherein the template consists of a promoter sequence and the downstream PCR primer comprises a template RNA sequence.

8. The process of claim 7 wherein the downstream PCR primer further contains a 3′ extension sequence.

9. The process of claim 1 wherein the small RNA consists of an siRNA.

10. The process of claim 9 wherein the siRNA consists of a hairpin siRNA.

11. The process of claim 1 wherein the small RNA consists of a sense strand or anti-sense strand of an siRNA.

12. The process of claim 6 wherein PCR amplification comprises sequential PCR amplification.

13. The process of claim 1 wherein the expression cassette consists of fewer than 1000 base pairs.

14. An expression cassette for expressing an RNA in a cell comprising: a linear DNA fragment consisting of a promoter, a sequence encoding the RNA, a transcription termination signal and a 3′ extension.

15. The expression cassette of claim 14 wherein the promoter consists of an RNA polymerase III promoter.

16. The expression cassette of claim 16 wherein the RNA polymerase III promoter consists of the U6 promoter.

17. The expression cassette of claim 16 wherein the RNA polymerase III promoter consists of the H1 promoter.

18. The expression cassette of claim 14 wherein the 3′ extension is greater than 5 nucleotides.

19. The expression cassette of claim 14 wherein the 3′ extension is greater than 20 nucleotides.

20. The expression cassette of claim 14 wherein the 3′ extension is greater than 50 nucleotides.

21. The process of claim 14 wherein the RNA consists of an siRNA.

22. The process of claim 22 wherein the siRNA consists of a hairpin siRNA.

23. The process of claim 14 wherein the RNA consists of a sense strand or anti-sense strand of an siRNA.

24. The expression cassette of claim 14 wherein the expression cassette consists of fewer than 1000 base pairs.

25. The expression cassette of claim 14 wherein the expression cassette consists of fewer than 500 base pairs.

26. A process for forming a linear expression cassette consisting of a promoter and a sequence encoding an RNA for expression of the RNA sequence in a cell comprising: PCR amplification of a promoter template wherein the upstream PCR primer consists of sequence that anneals upstream of or within the promoter and the downstream PCR primer consists of sequence encoding the RNA.

27. The process of claim 26 wherein the downstream PCR primer further consists of a transcription termination signal.

28. The process of claim 27 wherein the downstream PCR primer further consists of a 3′ extension.

29. The process of claim 28 wherein the 3′ extension is longer than 15 nucleotides.

30. The process of claim 29 wherein the 3′ extension is longer that 20 nucleotide.

31. The process of claim 26 wherein the sequence encoding the RNA consists of the sense strand or anti-strand of an siRNA

32. The process of claim 26 wherein the sequence encoding the siRNA consists of a hairpin siRNA.

33. A process for enhancing expression of an RNA from a linear expression cassette comprising: addition of a 3′ extension to the cassette downstream of a transcription termination signal.

34. The process of claim 29 wherein the 3′ extension is longer that 20 nucleotide.

35. The process of claim 28 wherein the 3′ extension is longer than 50 nucleotides.


SEQUENCE LISTING (31 sequences)

SEQ ID NO: 1 - DNA, 22 bp, Homo sapiens
cggtgtttcgtcctttccacaa

SEQ ID NO: 2 - DNA, 21 bp, Photinus pyralis
aagugcgcugcuggugccaac

SEQ ID NO: 3 - DNA, 74 bp, Artificial
  P. pyralis sequence plus vector sequence
agaaagtaatggctcgagagctggcaaggaaaaagttggcaccagcagcgcacggtgtttcgtcctttccacaa

SEQ ID NO: 4 - DNA, 74 bp, Artificial
  P. pyralis sequence plus vector sequence
agaaagtaatggctcgagagctggcaaggaaaaagtgcgctgctggtgccaacggtgtttcgtcctttccacaa

SEQ ID NO: 5 - DNA, 74 bp, Artificial
  expression cassette vector sequence
ttgtggaaaggacgaaacaccgtgcgctgctggtgccaactttttccttgccagctctcgagccattactttct

SEQ ID NO: 6 - DNA, 74 bp, Artificial
  expression cassette vector sequence
ttgtggaaaggacgaaacaccgttggcaccagcagcgcactttttccttgccagctctcgagccattactttct

SEQ ID NO: 7 - RNA, 21 nt, Artificial
  experimental control RNA oligonucleotide
gugcgcugcuggugccaacuu

SEQ ID NO: 8 - DNA, 21 bp, Photinus pyralis
guuggcaccagcagcgcacuu

SEQ ID NO: 9 - DNA, 10 bp, Homo sapiens
cttcctgtca

SEQ ID NO: 10 - DNA, 9 bp, Caenorhabditis elegans
ttcaagaga

SEQ ID NO: 11 - DNA, 31 bp, Photinus pyralis
aucagguggcucccgcugaauuggaauccau

SEQ ID NO: 12 - DNA, 113 bp, Artificial
  expression cassette vector sequence
ttgtggaaaggacgaaacaccggattccaattcagcgggagccacctgatgaagcttgatcgggtggctctcgctgagttggaatccatttttccttgccagctctcgagtcc

SEQ ID NO: 13 - RNA, 69 nt, Artificial
  haripin sequence
ggauuccaauucagcgggagccaccugaugaagcuugaucggguggcucucgcugaguuggaauccauu

SEQ ID NO: 14 - DNA, 113 bp, Artificial
  hairpin sequence
ggactcgagagctggcaaggaaaaatggattccaactcagcgagagccacccgatcaagcttcatcaggtggctcccgctgaattggaatccggtgtttcgtcctttccacaa

SEQ ID NO: 15 - DNA, 58 bp, Artificial
  hairpin sequence
caagcttcatcaggtggctcccgctgaattggaatccggtgtttcgtcctttccacaa

SEQ ID NO: 16 - DNA, 67 bp, Artificial
  hairpin sequence
ggactcgagagctggcaaggaaaaatggattccaactcagcgagagccacccgatcaagcttcatca

SEQ ID NO: 17 - DNA, 2552 bp, Artificial
  plasmid vector containing homo sapiens and bacterial sequence
gtcgagccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccctcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaactctagaccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgtcatttgcagggcttcccaaccttaccagagggcgccccagctggcaattccgcctgcaggtaccacatttgtagaggttttacttgctttaaaaaacctcccacatctccccctgaacctgaaacataaaatgaatgcaattgttgttgttgggccatggtggcggccgcgctagccggcagaaaagaaaatgcagaattttcaagtatttgggcttctaagctaagtttggggatagggttattttaatagtacagactccttcaaaaaataatatccccgaagaaaggaacagatcatcttcgggaaattgtttatctaagatgactgatacactagttcatacagataactaaagacaaaataatattcctgatttaaccattacttcggaacaccattggcctgggagcttgagagacgctactgagggtaatgcatataagctgaaaagcactcagtgggcatgaatagtgaaggaaaaaaaaagactttaagataaaagaaatatcatgagacaatgcttaaggcagaacaagaatttaaaaattaaacacacatacacacacacaccaagctaggaaagattaaccaaaggttatagagaagcctggtacgagatgagatggggtgtattgaagatggcattgtagctcatattagctgtagttaaacatttaagcttttaattaagaattcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcggtcttctgatatccaatgaagactcgagataggatccctgatct

SEQ ID NO: 18 - DNA, 29 bp, Mus musculus
gatggcattgtagctcatattagctgtag

SEQ ID NO: 19 - DNA, 360 bp, Artificial
  H. sapiens plus P. pyralis sequence
gatggcattgtagctcatattagctgtagttaaacatttaagcttttaattaagaattcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggattccaattcagcgggagccacctgatgaagcttg

SEQ ID NO: 20 - DNA, 415 bp, Artificial
  H. sapiens plus P. pyralis sequence
gatggcattgtagctcatattagctgtagttaaacatttaagcttttaattaagaattcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggattccaattcagcgggagccacctgatgaagcttgatcgggtggctctcgctgagttggaatccatttttccttgccagctctcgagtcc

SEQ ID NO: 21 - DNA, 29 bp, Homo sapiens
agggaattcaaggtcgggcaggaagaggg

SEQ ID NO: 22 - DNA, 402 bp, Mus musculus
gtcttgtctattgctggggttatagcgttaattctctgactagttttcttacctgctttcctcttgtcttttctaacctctcccccaaattagagcggcggtagtcctgtgcataatcagattagctcattgaactaaggaagccattcaatagctgtggactacaagcttaaatgtttaactacagctaatatgagctacaatgccatcttcaatacaccccatctcatctcgtaccaggcttctctataacctttggttaatctttcctagcttggtgtgtgtgtgtatgtgtgtttaatttttaaattcttgttctgccttaagcattgtctcatgatatttcttttatcttaaagtctttttttttccttcactattcatgcccactgagtgcttttc

SEQ ID NO: 23 - DNA, 67 bp, Artificial
  PCR primer with human U6 sequence
gaaggatccatctcccagctaaaaagtcatctgggtactcagggtcggtgtttcgtcctttccacaa

SEQ ID NO: 24 - DNA, 73 bp, Artificial
  PCR primer
gaaggatccatctcccagctaaaaagtctcggtggatctcgtatttcatgtcggtgtttcgtcctttccacaa

SEQ ID NO: 25 - DNA, 29 bp, Artificial
  PCR primer
agggaattcaaggtcgggcaggaagaggg

SEQ ID NO: 26 - DNA, 56 bp, Artificial
  PCR primer
ggcacactctggagagcatttaggcaagtggaattcaaggtcgggcaggaagaggg

SEQ ID NO: 27 - DNA, 83 bp, Artificial
  PCR primer
aaaaatgttcttctagaagccaagctcctccaagcttggaggagcttgacttccagaagaacggtgtttcgtcctttccacaa

SEQ ID NO: 28 - DNA, 82 bp, Artificial
  PCR primer
aaaaagcttgacttccagaagaacatttacaagcttgtaaatgttcttctagaagccaagcggtgtttcgtcctttccacaa

SEQ ID NO: 29 - DNA, 33 bp, Artificial
  PCR primer with mouse albumin gene sequence
ggttatagagaagcctggtacgagatgagatgg

SEQ ID NO: 30 - DNA, 22 bp, Artificial
  PCR primer
agagcgagtctttctgcacaca

SEQ ID NO: 31 - DNA, 21 bp, Artificial
  PCR primer
gtgtgcagaaagactcgctct